(19) United States
(12) Patent Application Publication
(10) Pub. No.: US 2021/0290680 A1
(43) Pub. Date: Sep. 23, 2021

(54) METHODS AND COMPOSITIONS FOR MODULATING CELLULAR AGING

(71) Applicant: Dorian Therapeutics, Inc., San Carlos, CA (US)

(72) Inventors: Benedetta NICOLIS DI ROBILANT, Redwood City, CA (US); Maddalena ADORNO, Redwood City, CA (US)

(73) Assignee: Dorian Therapeutics, Inc., San Carlos, CA (US)

(21) Appl. No.: 17/332,886

(22) Filed: May 27, 2021

Related U.S. Application Data
(63) Continuation of application No. PCT/US2020/048844, filed on Aug. 31, 2020.
(60) Provisional application No. 62/893,618, filed on Aug. 29, 2019.

(51) Int. Cl. A61K 35/17; C12N 15/86; C12N 15/113

(52) U.S. Cl. CPC A61K 35/17; C12N 15/86; C12N 15/113; C12N 2750/14143; C12N 2750/14121; C12N 2750/14122; C12N 2310/141

(57) ABSTRACT

The disclosure provides methods and compositions for modulating cellular aging comprising contacting a cell with an inhibitor of USP16. The disclosed methods may be used to improve the efficacy of cell-based therapies, including chimeric antigen receptor (CAR)-T cell therapies, engineered T cell receptor (TCR) therapies, natural killer (NK) cell therapies, hematopoietic stem cell-based therapies, and other adoptive cell therapies.

### CROSS REFERENCE TO RELATED APPLICATIONS

This application claims priority to U.S. provisional patent application No. 62/893,618, filed on Aug. 29, 2019, the contents of which are incorporated herein by reference in their entireties.

### INCORPORATION BY REFERENCE OF SEQUENCE LISTING

This application contains a Sequence listing which has been submitted in ASCII format via EFS-WEB and is hereby incorporated by reference in its entirety. Said ASCII copy, created on Aug. 28, 2020 is named DOTH_001_01WO_SeqList_25 and is 37 KB in size.

### BACKGROUND

Although cell-based therapies, such as chimeric antigen receptor (CAR)-T cell therapies and engineered T cell receptor (TCR) therapies, have shown great promise in the clinic, they are not always effective. For example, approximately 20% of patients treated with CD19 CAR-T cells do not respond to therapy. Additionally, solid tumors show very little response to redirected T cells, independently from the tumor antigen.

Cell-based therapies may be ineffective, for example, if the cells become aged, senescent, or exhausted before or shortly after they are administered to a subject. Cellular aging may be induced prematurely by irradiation, RAS, chemotherapy drugs and in actively dividing cells, for example, when cells to be used in cell-based therapies are expanded in vitro before administration to a subject. Cellular aging is a process typically accompanied by distinct phenotypic alterations, including chromatin remodeling, metabolic reprogramming, increased autophagy, and the implementation of a complex proinflammatory secretome. It may be associated with one or more of the following: decreased cell expansion, decreased in vitro expiration time, decreased in vivo persistence, decreased self-renewal ability, decreased self-renewal phenotype, increased cell exhaustion, decreased migration capability, increased production of reactive oxygen species (ROS), reduced mitochondrial membrane potential and/or glutathione content, decreased effector functions, decreased in vivo engraftment, decreased in vivo tumor killing, increased expression of senescence markers, increased CDKN2A expression, increased CDKN2D expression, increased CDKN1A expression, increased CDKN1B, decreased H2A or H2B ubiquitination, increased SA-β-Gal expression, increased telomere shortening, increased aging-associated markers and/or functions, increased aging-associated markers and/or functions, increased sensitivity to senolytics, increased SASP (senescence associated secretory phenotype), reduced cellular health, and/or increased necrosis/apoptosis (cell death).

There is a need to be able to modulate cellular aging, for example, to improve cell production for cell-based therapies.

### SUMMARY

Provided herein are compositions and methods that are useful for optimizing cells used in cell-based therapies. The compositions and methods described herein may be used to improve the efficacy of cell-based therapies, including CAR-T cell therapies, CAR-NK cell therapies, engineered TCR receptor therapies, redirected T cells, hematopoietic stem cell (HSC) therapies, and other adoptive cell therapies such as the use of tumor-infiltrating lymphocytes, NK cells, and regulatory T cells.

In some embodiments, the disclosure provides a method of modulating cellular aging comprising contacting a cell with an inhibitor of USP16, wherein the cell is a blood cell. In some embodiments, the blood cell is a genetically modified blood cell.

In some embodiments, the disclosure provides method of modulating cellular aging comprising contacting a cell with an inhibitor of USP16, wherein the cell is a T cell. In some embodiments, the T cell is a genetically modified T cell.

In some embodiments, the disclosure provides method of modulating cellular aging comprising contacting a cell with an inhibitor of USP16, wherein the cell is a NK cell. In some embodiments, the NK cell is a genetically modified NK cell.

In some embodiments, the disclosure provides method of modulating cellular aging comprising contacting a cell with an inhibitor of USP16, wherein the cell is a hematopoietic stem cell (HSC). In some embodiments, the hematopoietic stem cell is a genetically modified hematopoietic stem cell.

The methods described herein may have the effect of one or more of (a) maintaining or increasing cell expansion; (b) increasing in vitro expiration time; (c) increasing in vivo persistence; (d) preventing, delaying, or reversing the onset of senescence; (e) increasing or maintaining self-renewal ability; (f) increasing or maintaining self-renewal phenotypes; (g) reducing cell exhaustion; (h) maintaining migration capability; (i) reducing production of reactive oxygen species (ROS); (j) increasing effector functions; (k) increasing in vivo engraftment; (l) increasing in vivo tumor killing; (m) modulating the expression of senescence markers or aging-associated markers; (n) reducing CDKN2A, CDKN1A, CDKN2D, and/or γ-H2AX expression; (o) increasing cellular proliferation; (p) increasing H2A or H2B ubiquitination; (q) reducing SA-β-Gal expression; (r) reducing telomeres shortening; (s) enhancing signaling through the WNT pathway; (t) maintaining or increasing in vitro cytotoxicity; (u) maintaining or increasing Naïve or Central Memory phenotype; (v) modifying the type and quantity of released cytokines; (w) increasing the expression of stem cell markers; (x) reducing sensitivity to senolytics; (y) reducing SASP (senescence associated secretory phenotype); (z) reducing apoptosis; (aa) reducing necrosis; (bb) increasing mitochondrial membrane potential; and/or (cc) increasing cellular glutathione content. Also provided is a method of decreasing the expression of USP16 in a genetically modified blood cell comprising contacting the cell with an inhibitor of USP16.

Also provided is a method of preparing an immune cell for use in an immunotherapy application, comprising contacting the immune cell with an inhibitor of USP16. Such immunotherapies include chimeric antigen receptor (CAR)-based therapies, engineered T-cell-based therapies, hematopoietic stem cell-based therapies, and other adoptive cell therapies.

Also provided is a blood cell modified to downregulate expression of USP16.

Also provided is a method of treating a disease or disorder, comprising administering to a subject in need a therapeutically effective amount of a cell of the disclosure (e.g., a cell modified to downregulate USP16). In some embodiments, the treatment is autologous. In some embodiments, the treatment is allogeneic.

These and other aspects are addressed in more detail in the detailed description set forth below.

### BRIEF DESCRIPTION OF THE DRAWINGS

FIG. 1A-1B. FIG. 1A depicts T cells at different stages of differentiation, and the expression of cellular markers (CD45RA, CD45RO, CCR7, CD62L) associated with each stage.

FIG. 1B illustrates the phenotype associated with each stage of differentiation, as the cells progress from most stem-like (T_{N}: T naive) to least stem-like (T_{TE}: T terminal effector).

FIG. 2 is a graph showing relative expression of CDKN2A in naïve T cells (T_{N}), memory stem cells (T_{SCM}), central memory (T_{CM}), and effector memory cells (T_{EM}), as measured by in silico analysis of data published in Gattinoni L., et al, “A human memory T cell subset with stem cell-like properties,” Nature Medicine (2011).

FIG. 3A-3E are graphs showing relative expression of senescence markers BMI-1 (FIG. 3A), CDKN2A (FIG. 3B), CDKN2D (FIG. 3C) and exhaustion markers CTLA-4 (FIG. 3D) and PD-1 (FIG. 3E) in T cell subsets.

FIG. 4A-4C. FIG. 4A shows T cell expansion upon T cell activation at days 10, 20 and 30 after transduction with a lentivirus encoding either a shUSP16 or a non-targeting shRNA (Ctrl).

FIG. 4B shows viability of the cells after shUSP16 treatment, as determined using a Sytox staining assay. FIG. 4C shows the results of a T cell expiration assay. A statistically significant increase in persistence was observed for the cells treated with shUSP16.

FIG. 5 shows CD4/CD8 ratios at Day 20 after specific downregulation of USP16 expression. The downregulation of USP16 expression had no significant effect on CD4/CD8 ratio.

FIG. 6A-6C show cell survival in three different experiments wherein younger (day 0 to day 15 post-activation) and older (day 20 to day 40 after activation) cells were treated with the indicated amount of the senolytic drug Navitoclax.

FIG. 7A-7C show percent of dead cells at Day 12 (FIG. 7A) and Day 22 (FIG. 7B-7C) after cells were treated with the indicated amount of Navitoclax.

FIG. 8 shows the results of an in vitro limiting dilution assay at day 10 post activation. The solid grey line represents younger cells, and the solid black line represents older cells. Corresponding dotted lines show standard deviation.

FIG. 9 shows the results of an in vitro limiting dilution assay at day 20 post activation. The solid grey line represents the shUSP16 group, and the solid black line represents the control group. Corresponding dotted lines show standard deviation.

FIG. 10 shows the results of an in vitro limiting dilution assay at day 31 post activation. The solid grey line represents the shUSP16 group, and the solid black line represents the control group. Corresponding dotted lines show standard deviation.

FIG. 11A-11B demonstrate that Downregulation of USP16 expression increases the number of CD4⁺CD45RA⁺CD62L⁺ (FIG. 11A) and CD8⁺CD45RA⁺CD62L⁺ (FIG. 11B) cells in culture.

FIG. 12A-12B. FIG. 12A shows percent killing at different Effector:Target (E:T) ratios. No difference was observed between control cells (Ctrl) and USP16 downregulated cells. FIG. 12B shows IL-2 production upon antigen exposure. No effect on IL-2 production was observed after downregulation of USP16 expression.

FIG. 13A-13B. FIG. 13A is a graph showing expression of CDKN2A in CAR-T cells expressing a CD19.41BB CAR. For CDKN2A, values are normalized to CD19.CD28 CAR-T cells. FIG. 13B is a graph showing expression of BMI-1 in CAR-T cells with different costimulatory domains.

FIG. 14A-14B show that other shRNAs targeting USP16 effectively downregulate USP16 expression and inhibit senescence markers, while increasing cell proliferation.

FIG. 15 shows that down regulation of USP16 expression results in decreased expression of CDKN1A.

FIG. 16A-16B show that downregulation of USP16 expression enhances the activation of the WNT pathway.

FIG. 17A-17B show that downregulation of USP16 increases the frequency of stem cell memory T cells and function of the cells.

FIG. 18 shows that USP16 downregulation increases stem cell activity and functionality.

FIG. 19 shows that downregulation of USP16 expression decreases the expression of the exhaustion marker CD69.

FIG. 20 shows that T cells downregulating USP16 expression maintain the ability to kill tumor cells.

FIG. 21A-21B show that CRISPR-mediated knockout of USP16 was achieved by qPCR expression analyses.

FIG. 22 shows that CRISPR-mediated knockout of USP16 expression increases stem cell memory T cells.

FIG. 23 shows that CRISPR-mediated knockout of USP16 expression does not impair T cell-mediated killing.

FIG. 24 shows that USP16 expression was downregulated in CAR-T cells when the CAR construct co-expressed a shRNA targeting USP16.

FIG. 25 shows that downregulation of USP16 expression in CAR-T cells enhances CAR-T cell expansion.

FIG. 26A-26B show that downregulation of USP16 expression in CAR-T cells results in the decreased expression of the senescence markers CDKN1A and CDKN2A.

FIG. 27A-27B show that downregulation of USP16 expression in CAR-T cells enhances the signaling through the WNT pathway.

FIG. 28A-28B show that downregulation of USP16 expression in CAR-T cells increases stem cell memory T cell frequency.

FIG. 29 shows that downregulation of USP16 expression in CAR-T cells increases stem cell number and activity.

FIG. 30A-30B show that downregulation of USP16 expression in CAR-T cells increases killing and T cell expansion in a cytotoxicity assay.

FIG. 31A-31B show that downregulation of USP16 expression in CAR-T cells increases cellular health and reduces cellular and mitochondrial stress.

FIG. 32 shows that downregulation of USP16 expression in CAR-T cells reduces the expression of the exhaustion marker CD69.

FIG. 33 depicts the re-challenge experimental layout described in the examples.

FIG. 34 shows that downregulation of USP16 expression in CAR-T cells reduces the expression of exhaustion-related markers.

FIG. 35A-35B show that downregulation of USP16 expression significantly increases T cell killing capability upon multiple tumor challenges.

FIG. 36 shows the in vivo experimental design to evaluate the effect of the downregulation of USP16 expression in GD2.CAR-T cells on in vivo killing.

FIG. 37A-37B show significant increase of in vivo tumor killing when GD2.CAR-T cells downregulate USP16.

FIG. 38 shows the in vivo experimental design to evaluate the effect of the downregulation of USP16 expression in CD19.CAR-T cells on in vivo killing.

FIG. 39A-39B show significant increase of in vivo tumor killing when CD19.CAR-T cells downregulate USP16.

### DETAILED DESCRIPTION

The instant disclosure provides compositions and methods for modulating (e.g. suppressing or reverting) cellular aging. Specifically, it is shown herein that by reducing the expression or activity of a USP16 deubiquitinating enzyme (also known as Ubiquitin Specific Peptidase 16, Deubiquitinating Enzyme 16, Ubp-M, Ubiquitin carboxyl-terminal hydrolase 16) in a cell, one or more of the following effects may be achieved: (a) maintaining or increasing cell expansion; (b) increasing in vitro expiration time; (c) increasing in vivo persistence; (d) preventing, delaying, or reversing the onset of senescence; (e) increasing or maintaining self-renewal ability; (f) increasing or maintaining self-renewal phenotypes; (g) reducing cell exhaustion; (h) maintaining migration capability; (i) reducing production of reactive oxygen species (ROS); (j) increasing effector functions; (k) increasing in vivo engraftment; (1) increasing in vivo tumor killing; (m) modulating the expression of senescence markers or aging-associated markers; (n) reducing CDKN2A, CDKN1A, CDKN2D, and/or γ-H2AX expression; (o) increasing cellular proliferation; (p) increasing H2A or H2B ubiquitination; (q) reducing SA-β-Gal expression; (r) reducing telomeres shortening; (s) enhancing signaling through the WNT pathway; (t) maintaining or increasing in vitro cytotoxicity; (u) maintaining or increasing Naïve or Central Memory phenotype; (v) modifying the type and quantity of released cytokines; (w) increasing the expression of stem cell markers; (x) reducing sensitivity to senolytics; (y) reducing SASP (senescence associated secretory phenotype); (z) reducing apoptosis; (aa) reducing necrosis; (bb) increasing mitochondrial membrane potential; and/or (cc) increasing cellular glutathione content.

The compositions and methods of the instant disclosure may therefore be used to suppress or revert cellular aging in cells used in cell-based therapies including, but not limited to, CAR-T, CAR-NK, or CAR-macrophage cell therapies and other adoptive cell therapies (e.g., TCR-T cell therapies), stem cell therapies (e.g., hematopoietic stem cell therapies), and gene therapies, thereby increasing the effectiveness of such therapies.

Unless otherwise defined, all technical and scientific terms used herein have the same meaning as commonly understood by one of ordinary skill in the art to which this disclosure belongs. The terminology used in the detailed description herein is for the purpose of describing particular embodiments only and is not intended to be limiting.

All publications, patent applications, patents, GenBank or other accession numbers and other references mentioned herein are incorporated by reference herein in their entirety.

### Definitions

The below terms are used in the disclosure.

The singular forms “a,” “an” and “the” are intended to include the plural forms as well, unless the context clearly indicates otherwise.

Furthermore, the term “about” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even ±0.1% of the specified amount.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, the terms “reduce,” “decrease,” “lessen” and similar terms mean a decrease of at least about 10%, about 15%, about 20%, about 25%, about 35%, about 50%, about 75%, about 80%, about 85%, about 90%, about 95%, about 97%, or more.

As used herein, the terms “improve,” “increase,” “enhance,” and similar terms indicate an increase of at least about 10%, about 15%, about 20%, about 25%, about 50%, about 75%, about 100%, about 150%, about 200%, about 300%, about 400%, about 500%, or more.

As used herein “senolytic” refers to an agent that selectively induces death of senescent cells.

Unless the context indicates otherwise, it is specifically intended that the various features described herein can be used in any combination.

### Deubiquitinating Enzymes (DUBs) and Inhibitors Thereof

Ubiquitin regulates the degradation of proteins via the proteasome and lysosome, coordinates the cellular localization of proteins, activates and inactivates proteins, and modulates protein-protein interactions. Monoubiquitination of histones, in particular, has a role in chromatin remodeling, acting as an epigenetic regulator. Deubiquitinating enzymes (DUBs) such as USP16 cleave ubiquitin from proteins and other molecules.

USP16, encoded by the USP16 gene, is a histone H2A/H2B debuiquitinase. An exemplary amino acid sequence of full-length human USP16 (UNIPROT Accession No. Q9Y5T5) is provided below (SEQ ID NO: 1):

human USP16 amino acid sequence
SEQ ID NO: 1
MGKKRTKGKT VPIDDSSETL EPVCRHIRKG LEQGNLKKAL
VNVEWNICQD CKTDNKVKDK AEEETEEKPS VWLCLKCGHQ
GCGRNSQEQH ALKHYLTPRS EPHCLVLSLD NWSVWCYVCD
NEVQYCSSNQ LGQVVDYVRK QASITTPKPA EKDNGNIELE
NKKLEKESKN EQEREKKENM AKENPPMNSP CQITVKGLSN
LGNTCFFNAV MQNLSQTPVL RELLKEVKMS GTIVKIEPPD
LALTEPLEIN LEPPGPLTLA MSQFLNEMQE TKKGVVTPKE
LFSQVCKKAV RFKGYQQQDS QELLRYLLDG MRAEEHQRVS
KGILKAFGNS TEKLDEELKN KVKDYEKKKS MPSFVDRIFG
GELTSMIMCD QCRTVSLVHE SFLDLSLPVL DDQSGKKSVN
DKNLKKTVED EDQDSEEEKD NDSYIKERSD IPSGTSKHLQ
KKAKKQAKKQ AKNQRRQQKI QGKVLHLNDI CTIDHPEDSE
YEAEMSLQGE VNIKSNHISQ EGVMHKEYCV NQKDLNGQAK
MIESVTDNQK STEEVDMKNI NMDNDLEVLT SSPTRNLNGA
YLTEGSNGEV DISNGFKNLN LNAALHPDEI NIEILNDSHT
PGTKVYEVVN EDPETAFCTL ANREVFNTDE CSIQHCLYQF
TRNEKLRDAN KLLCEVCTRR QCNGPKANIK GERKHVYTNA
KKQMLISLAP PVLTLHLKRF QQAGFNLRKV NKHIKFPEIL
DLAPFCTLKC KNVAEENTRV LYSLYGVVEH SGTMRSGHYT
AYAKARTANS HLSNLVLHGD IPQDFEMESK GQWFHISDTH
VQAVPTTKVL NSQAYLLFYE RIL

Several additional USP16 isoforms are also known. See GenBank Accession Nos. NM_006447.2 (SEQ ID NO: 3), NM_006447.3 (SEQ ID NO: 4), NM_001001992.1 (SEQ ID NO: 5), and NM_001032410.1 (SEQ ID NO: 6). See also, Uniprot Accession No. H9KVB6 (SEQ ID NO: 7).

SEQ ID NO: 3: USP16 protein variant
MGKKRTKGKTVPIDDSSETLEPVCRHIRKGLEQGNLKKALVNVEWNICQ
DCKTDNKVKDKAEEETEEKPSVWLCLKCGHQGCGRNSQEQHALKHYLTP
RSEPHCLVLSLDNWSVWCYVCDNEVQYCSSNQLGQVVDYVRKQASITTP
KPAEKDNGNIELENKKLEKESKNEQEREKKENMAKENPPMNSPCQITVK
GLSNLGNTCFFNAVMQNLSQTPVLRELLKEVKMSGTIVKIEPPDLALTE
PLEINLEPPGPLTLAMSQFLNEMQETKKGVVTPKELFSQVCKKAVRFKG
YQQQDSQELLRYLLDGMRAEEHQRVSKGILKAFGNSTEKLDEELKNKVK
DYEKKKSMPSFVDRIFGGELTSMIMCDQCRTVSLVHESFLDLSLPVLDD
QSGKKSVNDKNLKKTVEDEDQDSEEEKDNDSYIKERSDIPSGTSKHLQK
KAKKQAKKQAKNQRRQQKIQGKVLHLNDICTIDHPEDSEYEAEMSLQGE
VNIKSNHISQEGVMHKEYCVNQKDLNGQAKMIESVTDNQKSTEEVDMKN
INMDNDLEVLTSSPTRNLNGAYLTEGSNGEVDISNGFKNLNLNAALHPD
EINIEILNDSHTPGTKVYEVVNEDPETAFCTLANREVFNTDECSIQHCL
YQFTRNEKLRDANKLLCEVCTRRQCNGPKANIKGERKHVYTNAKKQMLI
SLAPPVLTLHLKRFQQAGFNLRKVNKHIKFPEILDLAPFCTLKCKNVAE
ENTRVLYSLYGVVEHSGTMRSGHYTAYAKARTANSHLSNLVLHGDIPQD
FEMESKGQWFHISDTHVQAVPTTKVLNSQAYLLFYERIL
SEQ ID NO: 4: USP16 protein variant
MGKKRTKGKTVPIDDSSETLEPVCRHIRKGLEQGNLKKALVNVEWNICQ
DCKTDNKVKDKAEEETEEKPSVWLCLKCGHQGCGRNSQEQHALKHYLTP
RSEPHCLVLSLDNWSVWCYVCDNEVQYCSSNQLGQVVDYVRKQASITTP
KPAEKDNGNIELENKKLEKESKNEQEREKKENMAKENPPMNSPCQITVK
GLSNLGNTCFFNAVMQNLSQTPVLRELLKEVKMSGTIVKIEPPDLALTE
PLEINLEPPGPLTLAMSQFLNEMQETKKGVVTPKELFSQVCKKAVRFKG
YQQQDSQELLRYLLDGMRAEEHQRVSKGILKAFGNSTEKLDEELKNKVK
DYEKKKSMPSFVDRIFGGELTSMIMCDQCRTVSLVHESFLDLSLPVLDD
QSGKKSVNDKNLKKTVEDEDQDSEEEKDNDSYIKERSDIPSGTSKHLQK
KAKKQAKKQAKNQRRQQKIQGKVLHLNDICTIDHPEDSEYEAEMSLQGE
VNIKSNHISQEGVMHKEYCVNQKDLNGQAKMIESVTDNQKSTEEVDMKN
INMDNDLEVLTSSPTRNLNGAYLTEGSNGEVDISNGFKNLNLNAALHPD
EINIEILNDSHTPGTKVYEVVNEDPETAFCTLANREVFNTDECSIQHCL
YQFTRNEKLRDANKLLCEVCTRRQCNGPKANIKGERKHVYTNAKKQMLI
SLAPPVLTLHLKRFQQAGFNLRKVNKHIKFPEILDLAPFCTLKCKNVAE
ENTRVLYSLYGVVEHSGTMRSGHYTAYAKARTANSHLSNLVLHGDIPQD
FEMESKGQWFHISDTHVQAVPTTKVLNSQAYLLFYERIL
SEQ ID NO: 5: USP16 protein variant
MGKKRTKGKTVPIDDSSETLEPVCRHIRKGLEQGNLKKALVNVEWNICQ
DCKTDNKVKDKAEEETEEKPSVWLCLKCGHQGCGRNSQEQHALKHYLTP
RSEPHCLVLSLDNWSVWCYVCDNEVQYCSSNQLGQVVDYVRKQASITTP
KPEKDNGNIELENKKLEKESKNEQEREKKENMAKENPPMNSPCQITVKG
LSNLGNTCFFNAVMQNLSQTPVLRELLKEVKMSGTIVKIEPPDLALTEP
LEINLEPPGPLTLAMSQFLNEMQETKKGVVTPKELFSQVCKKAVRFKGY
QQQDSQELLRYLLDGMRAEEHQRVSKGILKAFGNSTEKLDEELKNKVKD
YEKKKSMPSFVDRIFGGELTSMIMCDQCRTVSLVHESFLDLSLPVLDDQ
SGKKSVNDKNLKKTVEDEDQDSEEEKDNDSYIKERSDIPSGTSKHLQKK
AKKQAKKQAKNQRRQQKIQGKVLHLNDICTIDHPEDSEYEAEMSLQGEV
NIKSNHISQEGVMHKEYCVNQKDLNGQAKMIESVTDNQKSTEEVDMKNI
NMDNDLEVLTSSPTRNLNGAYLTEGSNGEVDISNGFKNLNLNAALHPDE
INIEILNDSHTPGTKVYEVVNEDPETAFCTLANREVFNTDECSIQHCLY
QFTRNEKLRDANKLLCEVCTRRQCNGPKANIKGERKHVYTNAKKQMLIS
LAPPVLTLHLKRFQQAGFNLRKVNKHIKFPEILDLAPFCTLKCKNVAEE
NTRVLYSLYGVVEHSGTMRSGHYTAYAKARTANSHLSNLVLHGDIPQDF
EMESKGQWFHISDTHVQAVPTTKVLNSQAYLLFYERIL
SEQ ID NO: 6: USP16 protein variant
MGKKRTKGKTVPIDDSSETLEPVCRHIRKGLEQGNLKKALVNVEWNICQ
DCKTDNKVKDKAEEETEEKPSVWLCLKCGHQGCGRNSQEQHALKHYLTP
RSEPHCLVLSLDNWSVWCYVCDNEVQYCSSNQLGQVVDYVRKQASITTP
KPAEKDNGNIELENKKLEKESKNEQEREKKENMAKENPPMNSPCQITVK
GLSNLGNTCFFNAVMQNLSQTPVLRELLKEVKMSGTIVKIEPPDLALTE
PLEINLEPPGPLTLAMSQFLNEMQETKKGVVTPKELFSQVCKKAVRFKG
YQQQDSQELLRYLLDGMRAEEHQRVSKGILKAFGNSTEKLDEELKNKVK
DYEKKKSMPSFVDRIFGGELTSMIMCDQCRTVSLVHESFLDLSLPVLDD
QSGKKSVNDKNLKKTVEDEDQDSEEEKDNDSYIKERSDIPSGTSKHLQK
KAKKQAKKQAKNQRRQQKIQGKVLHLNDICTIDHPEDSEYEAEMSLQGE
VNIKSNHISQEGVMHKEYCVNQKDLNGQAKMIESVTDNQKSTEEVDMKN
INMDNDLEVLTSSPTRNLNGAYLTEGSNGEVDISNGFKNLNLNAALHPD
EINIEILNDSHTPGTKVYEVVNEDPETAFCTLANREVFNTDECSIQHCL
YQFTRNEKLRDANKLLCEVCTRRQCNGPKANIKGERKHVYTNAKKQMLI
SLAPPVLTLHLKRFQQAGFNLRKVNKHIKFPEILDLAPFCTLKCKNVAE
ENTRVLYSLYGVVEHSGTMRSGHYTAYAKARTANSHLSNLVLHGDIPQD
FEMESKGQWFHISDTHVQAVPTTKVLNSQAYLLFYERIL
SEQ ID NO: 7: USP16 protein variant
MGKKRTKGKTVPIDDSSETLEPVCRHIRKGLEQGNLKKALVNVEWNICQ
DCKTDNKVKDKAEEETEEKPSVWLCLKCGHQGCGRNSQEQHALKHYLTP
RSEPHCLVL

Provided herein are inhibitors of USP16. Such inhibitors may reduce the expression and/or activity of USP16 in a cell. Exemplary inhibitors of USP16 include, for example, nucleic acids, proteins, small molecules, or large molecules.

In some embodiments, the inhibitor of USP16 is a RNA-guided nuclease, such as a Cas nuclease, or a nucleic acid encoding the same. In some embodiments, the Cas nuclease is a Cas9 nuclease, a Cas12(a) nuclease (Cpf1), a Cas12b nuclease, a Cas12c nuclease, a TrpB-like nuclease, a Cas13a nuclease (C2c2), a Cas13b nuclease, a Cas14 nuclease, a CasX nuclease, a CasY nuclease, or modified or truncated variants thereof. In some embodiments, the Cas9 nuclease is isolated or derived from S. pyogenes or S. aureus. Cas nucleases bind to a guide RNA (e.g., a single-molecule or dual-molecule gRNA), which binds to a target nucleic acid sequence. Single-molecule gRNAs (e.g., sgRNAs) typically comprise a spacer sequence that is complimentary to a target DNA sequence of interest, and a scaffold sequence that binds to the Cas nuclease. In some embodiments, the spacer sequence targets a specific site in the USP16 gene, such as an intron or an exon of the USP16 gene. In some embodiments, the spacer sequence targets non-coding regions, including, but not limited to, a promoter, an enhancer, or other untranslated regions (e.g. 5′UTR or 3′UTR) of the USP16 gene.

In some embodiments, the inhibitor of USP16 is a transcription activator-like effector nuclease (TAL nuclease), or one or more nucleic acids encoding the same. A TAL nuclease may comprise a TAL effector DNA-binding domain fused to a DNA cleavage domain. The TAL nuclease may target the USP16 gene, such as in intron or an exon of the USP16 gene. In some embodiments, the TAL nuclease targets non-coding regions, including, but not limited to, a promoter, an enhancer, or other untranslated regions (e.g. 5′UTR or 3′UTR) of the USP16 gene.

In some embodiments, the inhibitor of USP16 is a zinc (Zn) finger nuclease, or one or more nucleic acids encoding the same. A Zn finger nuclease may comprise a zinc finger DNA-binding domain fused to a DNA cleavage domain (e.g., FokI or a variant thereof). The Zn finger nuclease may target the USP16 gene, such as in intron or an exon of the USP16 gene. In some embodiments, the Zn finger nuclease targets non-coding regions, including, but not limited to, a promoter, an enhancer, or other untranslated regions (e.g. 5′UTR or 3′UTR) of the USP16 gene.

In some embodiments, the inhibitor is a RNAi molecule. For example, the inhibitor may be a small hairpin RNA (shRNA), a small interfering RNA (siRNA), a microRNA, or an asymmetric interfering RNA. In some embodiments, the inhibitor is a shRNA targeting a sequence of the USP16 gene. In some embodiments, the inhibitor is a shRNA targeting the following sequence of the USP16 gene: 5′-TCCAGAAGGAATATCACTT-3′ (SEQ ID NO: 2), 5′-GACTGTAAGACTGACAATAAA-3′ (SEQ ID NO: 8) and 5′-TATATCAGTTCACCCGTAAT-3′ (SEQ ID NO: 9) or a sequence at least 90%, at least 95%, at least 96%, at least 97%, at least 98%, or at least 99% identical thereto.

In some embodiments, the inhibitor of USP16 is selected from an antisense molecule, a phosphorothioate oligonucleotide, a DNA-RNA chimera, a morpholino oligo, a lhRNA, a miRNA embedded shRNA, a small internally segmented RNA, an antibody, and an exosome.

In some embodiments, an inhibitor of USP16 (e.g., a small molecule inhibitor of USP16) is used in combination with one or more other agents. In some embodiments, a first inhibitor of USP16 is used in combination with a second inhibitor of USP16. In some embodiments, an inhibitor of USP16 is used in combination with one or more WNT agonists. In some embodiments, an inhibitor of USP16 is used in combination with one or more R-spondin (Rspo) agonists. In some embodiments, an inhibitor of USP16 is used in combination with an anti-exhaustion therapy, (e.g. PD1 inhibition therapy (e.g. anti-PD1 therapy), e.g. CTLA4 inhibition therapy (e.g. anti-CTLA4 therapy). In some embodiments, an inhibitor of USP16 and a second agent are used simultaneously. In some embodiments, an inhibitor of USP16 and a second agent are used sequentially.

In some embodiments, USP16 inhibition refers to knockdown of USP16 expression (also interchangeably referred to herein as downregulation, decreasing, silencing, etc. of expression). The knockdown need not be a total knockout of expression, and so in some embodiments, the inhibitor of USP16 may reduce expression of USP16 by at least 10%, at least 20%, at least 30%, at least 40%, at least 50%, at least 60%, at least 70%, at least 80%, at least 90%, at least 95%, or at least 99%. In some embodiments, the inhibitor of USP16 may completely eliminate expression of USP16 (e.g. knockout).

In some embodiments, USP16 inhibition refers to knockdown of USP16 activity (also interchangeably referred to herein as downregulation, decreasing, silencing, etc. of activity). In some embodiments, the inhibitor of USP16 may reduce activity of USP16 by at least 10%, at least 20%, at least 30%, at least 40%, at least 50%, at least 60%, at least 70%, at least 80%, at least 90%, at least 95%, or at least 99%. In some embodiments, the inhibitor of USP16 may completely eliminate the activity of USP16.

In some embodiments, USP16 inhibition refers to a modification of the USP16 gene, which renders it inoperative (e.g., a knockout of the USP16 gene or specific base mutations (e.g. mutations in the catalytic site).

In some embodiments, after a USP16 inhibitor is contacted with cells, USP16 expression and/or activity is inhibited in at least 10%, at least 20%, at least 30%, at least 40%, at least 50%, at least 60%, at least 70%, at least 80%, at least 90%, at least 95%, at least 99%, or 100% of the cells.

### Cells

Provided herein methods of contacting cells and methods of modifying cells to downregulate expression of USP16. In some embodiments, the cell is isolated or derived from a reptile, an amphibian, a bird, a mammal, or a fish. In some embodiments, the cell is a primate cell. In some embodiments, the cell is a human cell. In some embodiments, the cell is a non-human primate cell, e.g. a monkey cell.

The cells of the disclosure may be contacted and modified in vivo or in vitro. In embodiments wherein the cell is in vitro, the cell may be a primary cell or an immortalized cell. The cell may be wildtype, may be genetically modified, or may comprise one or more genetic mutations. In some embodiments, the genome of the cell has one copy, two copies, or three copies of the USP16 gene. In some embodiments, the cell may express high levels of USP16, or may express low levels of USP16. As used herein, a “high” level of USP16 refers to a level of USP16 that is increased at least 10%, at least 20%, at least 30%, at least 40%, at least 50%, at least 60%, at least 70%, at least 80%, at least 90%, or at least 100% compared to wildtype levels. A “low” level of USP refers to a level of USP16 that is decreased at least 10%, at least 20%, at least 30%, at least 40%, at least 50%, at least 60%, at least 70%, at least 80%, at least 90%, or at least 100% compared to wildtype levels. A low or high level of USP16 expression may be induced by genetic modification, aging, etc.

The cell may be a wildtype cell or an engineered (e.g. genetically modified) cell to be used for a cell-based therapy (e.g., a chimeric antigen receptor-based cell therapy, a HSC therapy or a engineered TCR therapy).

In some embodiments, the cell is a blood cell. The blood cell may be a hematopoietic stem cells, a common myeloid or lymphocyte progenitor or a cell differentiating from them, including a red blood cell, a white blood cell, or a platelet. In some embodiments, the blood cell is an immune cell such as a T cell, a B cell, a dendritic cell, a monocyte, a macrophage, an eosinophil, a basophil, a neutrophil, or a natural killer cell (NK cell; which is different than the below mentioned natural killer T cell).

In embodiments wherein the cell is a T cell, the T cell may be a CD4+ T cell or a CD8+ T cell. For example, the T cell may be a cytotoxic T cell, a terminal effector T cell, a memory or a central memory T cell, a naïve T cell, a regulatory T cell, a natural killer T cell, a gamma-delta T cell, a cytokine-induced killer (CIK) T cell, or a tumor infiltrating lymphocyte.

In embodiments wherein the cell is a NK (natural killer) cell, the NK cell may be a NK^{tolerant} (e.g. CD56^{bright} or CD27⁻CD11b⁻ NK cell), NK^{cytotoxic} (e.g. CD56^{dim} or CD27⁻CD11b⁺ NK cell), NK^{regulatory} (e.g. CD56^{bright} or CD27⁺ NK cell) or NKT (CD56⁻ or CD3⁺CD56⁻).

In some embodiments, the cell is an iPSC-derived NK cell. In some embodiments, the cell is a stem cell. The stem cell may be, for example, an induced pluripotent stem cell (iPSC). In some embodiments, the cell is a hematopoietic stem cell (HSC). In some embodiments, the cell is a HSC to be used for a HSC therapy. For example, the HSC may be modified to express one or more therapeutic genes or a chimeric antigen receptor (CAR) or a T-cell receptor (TCR).

In some embodiments, the cell is not a stem cell. In some embodiments, the cell is not a blood stem cell. In some embodiments, the cell is not a lymphoid or myeloid precursor cell. In some embodiments, the cell is not a hematopoietic stem cell.

Any of the cells described above may be genetically modified in some embodiments of the disclosure. As used herein, the term “genetically modified cell” refers to a cell that is genetically engineered to stably or transiently express a DNA segment encoding a protein or a RNA transcript. In some embodiments, a genetically modified cell is not modified to overexpress USP16. In some embodiments, the cell is genetically modified to express or overexpress a protein of interest. In some embodiments, the cell is genetically modified to downregulate (knockdown or knockout) expression of a protein of interest. In some embodiments, the cell is genetically modified to overexpress a protein of interest that is already expressed in the cell, or express it at a time/context when it is typically not expressed. In some embodiments, the cell is genetically modified to express an exogenous protein, e.g. a protein that is not normally expressed by the cell.

Any of the cells described above may be cells to be expanded in vitro for cellular therapy. For example, the cell may be expanded about 20-fold, about 50-fold, about 100-fold, about 250-fold, about 500-fold, about 750-fold, about 1000-fold or more before being administered to a subject, In some embodiments, a genetically modified cell may be expanded in vitro before it is administered to a subject in need thereof.

In some embodiments, the cell is a genetically modified blood cell. In some embodiments, the genetically modified blood cell is genetically modified to express on its surface a chimeric antigen receptor or a T-cell receptor (TCR). In some embodiments, the cells are prepared for an adoptive cell therapy application which is allogeneic. In some embodiments, the cells are prepared for an adoptive cell therapy application which is autologous.

In some embodiments, a genetically modified blood cell may be a cell modified to express a chimeric antigen receptor (CAR, eg. a CAR-T cell, CAR-macrophage, or a CAR-NK cell). The CAR-modified cell may express a chimeric antigen receptor that recognizes one or more antigens on the surface of a target cell. For example, the CAR-modified cell may express a chimeric antigen receptor that recognizes, for example, CD19, CD20, CD22, CD30, CD33, CD70, CD123, CD138, CD171, glypican-3, kappa immunoglobulin, ROR1, GD2, CD44v6, HER2, NY-ESO-1, BCMA, CD22, MSLN, CEA, EGFR, EGFRvIII, VEGFR2, IL-13, IL13Rα2, Lewis Y antigen, mesothelin, FAP, and/or PSMA. In some embodiment the CAR is a dual-targeting CAR, an inhibitory CAR, an inducible CAR, a synNotch CAR, an iCAR, a drug-inducible CAR, or an adapter CAR. In some embodiments the CAR can co-express a cytokine or a cytokine receptor (e.g. IL15 or IL15R), or a suicide gene. In some embodiments, the CAR can express anti-exhaustion proteins (e.g. PD-1/PDL-1 Fab) or shRNA/siRNA/gRNA to regulate exhaustion markers (e.g. PD-1 shRNA) and reduce the expression of inhibitory receptors.

In other embodiments, the cell is a genetically modified blood cell and is modified to express a T-cell receptor, for example for use in T-cell receptor (TCR-T cell) therapies. In some embodiments, the TCR editing can be complete or partial editing.

In some embodiments, the cell is an autologous cell; in other embodiments, the cell is an allogenic cell. In some embodiments, when preparing the cell for an autologous or allogeneic therapy, additional gene modifications may be carried out.

Methods for Downregulating the Expression and/or Activity of USP16 in a cell

Provided herein are methods of decreasing (interchangeably referred to herein as downregulating, knocking down, silencing, inhibiting) the expression and/or activity of USP16 in a cell, the methods comprising contacting an inhibitor of USP16 with the cell. It is noted that the downregulation of expression and/or activity may be transient or stable. These methods may be performed in vitro, or in vivo.

Decreasing the expression and/or activity of USP16 in cells may have one or more of the following effects: (a) maintaining or increasing cell expansion; (b) increasing in vitro expiration time; (c) increasing in vivo persistence; (d) preventing, delaying, or reversing the onset of senescence; (e) increasing or maintaining self-renewal ability; (f) increasing or maintaining self-renewal phenotypes; (g) reducing cell exhaustion; (h) maintaining migration capability; (i) reducing production of reactive oxygen species (ROS); (j) increasing effector functions; (k) increasing in vivo engraftment; (l) increasing in vivo tumor killing; (m) modulating the expression of senescence markers or aging-associated markers; (n) reducing CDKN2A, CDKN1A, CDKN2D, and/or γ-H2AX expression; (o) increasing cellular proliferation; (p) increasing H2A or H2B ubiquitination; (q) reducing SA-β-Gal expression; (r) reducing telomeres shortening; (s) enhancing signaling through the WNT pathway; (t) maintaining or increasing in vitro cytotoxicity; (u) maintaining or increasing Naïve or Central Memory phenotype; (v) modifying the type and quantity of released cytokines; (w) increasing the expression of stem cell markers; (x) reducing sensitivity to senolytics; (y) reducing SASP (senescence associated secretory phenotype); (z) reducing apoptosis; (aa) reducing necrosis; (bb) increasing mitochondrial membrane potential; and/or (cc) increasing cellular glutathione content.

In some embodiments wherein the cell is a T cell, reducing the expression and/or activity of USP16 in the cell may reduce T cell exhaustion. Accordingly, the compositions and methods described herein may be used to improve the efficacy of cell-based therapies, including CAR-T therapies and engineered TCR-T cell therapies.

In some embodiments, the disclosure provides a method of modulating cellular aging the method comprising contacting a cell with an inhibitor of USP16. The method may result in one or more of: ((a) maintaining or increasing cell expansion; (b) increasing in vitro expiration time; (c) increasing in vivo persistence; (d) preventing, delaying, or reversing the onset of senescence; (e) increasing or maintaining self-renewal ability; (f) increasing or maintaining self-renewal phenotypes; (g) reducing cell exhaustion; (h) maintaining migration capability; (i) reducing production of reactive oxygen species (ROS); (j) increasing effector functions; (k) increasing in vivo engraftment; (l) increasing in vivo tumor killing; (m) modulating the expression of senescence markers or aging-associated markers; (n) reducing CDKN2A, CDKN1A, CDKN2D, and/or γ-H2AX expression; (o) increasing cellular proliferation; (p) increasing H2A or H2B ubiquitination; (q) reducing SA-β-Gal expression; (r) reducing telomeres shortening; (s) enhancing signaling through the WNT pathway; (t) maintaining or increasing in vitro cytotoxicity; (u) maintaining or increasing Naïve or Central Memory phenotype; (v) modifying the type and quantity of released cytokines; (w) increasing the expression of stem cell markers; (x) reducing sensitivity to senolytics; (y) reducing SASP (senescence associated secretory phenotype); (z) reducing apoptosis; (aa) reducing necrosis; (bb) increasing mitochondrial membrane potential; and/or (cc) increasing cellular glutathione content.

In some embodiments, the cell is a blood cell. In some embodiments, the cell is a HSC. In some embodiments, the cell is an immune cell. In some embodiments, the cell is a T cell. In some embodiments, the cell is a NK cell. In some embodiments, the cell is a genetically modified cell, such as a genetically modified T cell, genetically modified NK cell, or a genetically modified HSC. In some embodiments wherein the cell is a T cell (e.g., a genetically modified T cell), reducing the activity of USP16 in the cell may reduce T cell exhaustion.

In some embodiments, provided is a method for preparing an immune cell for use in an immunotherapy application, and the method comprises contacting the immune cell with an inhibitor of USP16. In some embodiments, the immunotherapy application is a CAR-based therapy (e.g. CAR-T cell, CAR-macrophage, or CAR-NK cell therapy), an engineered TCR therapy, or other adoptive cell therapy such as the use of tumor-infiltrating lymphocytes, regulatory T cells, and redirected T cells, a stem cell therapy (e.g., HSC therapy), or a gene therapy. In some embodiments, the immunotherapy is autologous. In some embodiments, the immunotherapy is allogenic.

In some embodiments, the inhibitor of USP16 is contacted with the immune cell during in vitro expansion of a population of cells comprising the immune cell. In some embodiments, the immune cell is contacted with the inhibitor of USP16 before the cell is administered to the subject, for example about 4 hours, about 12 hours, about 24 hours, about 26 hours, or about 48 hours before the cell is administered to the subject. In some embodiments, the immune cell is contacted with the inhibitor of USP16 about 3 to about 21 days, about 7 to about 21 days, or about 7 to about 14 days before the cell is administered to the subject. In some embodiments, the immune cell is contacted with the inhibitor of USP16 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days before the cell is administered to the subject. In some embodiments, the immune cell is contacted with the inhibitor of USP16 after the cell is administered to the subject, for example about 4 hours, about 12 hours, about 24 hours, about 26 hours, or about 48 hours after the cell is administered to the subject. In some embodiments, the immune cell is administered to the subject concurrently with the inhibitor of USP16.

Effects of Downregulating the Expression and/or Activity of USP16 in a cell

As is noted in the above section, an observed or expected result of downregulating the expression and/or activity of USP16 in the cells of the disclosure is the associated maintenance or increase in cell expansion. As referred to herein, cell expansion generally refers to the in vitro process of cell proliferation, typically the cells are taken from an organism or tissue prior to expansion. In some embodiments, such increase can be an increase of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated increase in cellular proliferation. As referred to herein, cellular proliferation is the process that results in an increase of the number of cells, indicative of a balance between cell divisions and cell loss through cell death or differentiation. Cellular proliferation can be measured in vitro or in vivo. In some embodiments, such increase can be an increase of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated increase in in vitro expiration time. In some embodiments, such increase can be an increase of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated increase in in vivo persistence. In some embodiments, such increase can be an increase of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated prevention, delay, or reversal in the onset of senescence. In some embodiments, such prevention, delay, or reversal may be by about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated increase in or maintenance of self-renewal ability. In some embodiments, such increase can be an increase of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated increase in or maintenance of self-renewal phenotypes. In some embodiments, such increase can be an increase of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an expected result of downregulating the expression and/or activity of USP16 in the cells of the disclosure is the associated reduction in cell exhaustion. Cellular exhaustion may be assessed in a number of ways, and in some embodiments, the expression levels of exhaustion markers may be assessed. Exhaustion markers include, but are not limited to PD-1, Lag3, CTLA4, CD69, CD39, TIM-3, TOX2, TIGIT, CD160, 2B4, and. BTLA. In some embodiments, such reduction of exhaustion, or the expression levels of exhaustion markers can be a reduction of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated maintaining migration capability.

As is noted in the above section, an observed or expected result of downregulating the expression and/or activity of USP16 in the cells of the disclosure is the associated reduction in the production of reactive oxygen species (ROS). In some embodiments, such reduction can be a reduction of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated increase in effector functions, e.g. T-cell effector functions. In some embodiments, such increase can be an increase of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated increase in in vivo engraftment. In some embodiments, such increase can be an increase of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated increase in in vivo tumor killing. In some embodiments, such increase can be an increase of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated modulation in the expression of senescence markers. As used herein, a senescence marker is a gene, protein, metabolite or epigenetic marker that can increase or decrease during cellular aging. Senescence markers include, but are not limited to CDKN2A, CDKN1A, CDKN2B, CDKN2D, p27, p53, LaminB1, BMI-1, γ-H2AX, FOXO3, FOXO1, FOXM1, CCND1, IL6, IL8, STAT3, STATE, CDK6 and genes related to glycolysis. It is noted that T cells exhibit a change in the genetic profile of additional genes (in addition to the aforementioned ones) during cellular aging and senescence, and thus with respect to a senescence markers in T cells, also included are CD27, CD28, CD57, CD160, CD27, KLRG1 and CD138. In some embodiments, such modulation can be an increase or decrease of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300% in the levels of expression.

As is noted in the above section, an observed or expected result of downregulating the expression and/or activity of USP16 in the cells of the disclosure is the associated reduction of CDKN2A, CDKN1A, CDKN2D, and/or γ-H2AX expression. In some embodiments, such reduction can be a reduction of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated increase in H2A or H2B ubiquitination. In some embodiments, such increase can be an increase of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated reduction in SA-β-Gal expression. In some embodiments, such reduction can be a reduction of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated reduction of telomere shortening. In some embodiments, such reduction can be a reduction of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated enhancement (increase) in signaling through the WNT pathway. The enhancement of signaling through the WNT pathway can be detected by the expression levels of several genes, including but not limited to WNT3A, WNT11, WNT10, WNT5a, TCF7, LEF1, AXIN2, CTNBB1, NOTCH, Fzd, LPR5/6, LGR4/5/6, APC, GSK3, c-myc, c-jun, and Cyclin D1 (CCND1). Accordingly, as used herein, enhancing of signaling through the Wnt pathway may, in some embodiments be detected by assessing the expression levels of one or more of the aforementioned genes. In some embodiments, such increase in signaling/expression of markers can be an increase of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated maintenance or increase in in vitro cytotoxicity. In some embodiments, such increase can be an increase of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated maintenance or increase in a Naïve or Central Memory phenotype. In some embodiments, such increase can be an increase of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in this section, an observed or expected result of downregulating the expression and/or activity of USP16 in the cells of the disclosure is the associated modification in the type and quantity of released cytokines. Accordingly, in some embodiments, the cells of the disclosure may reduce the production of cytokines related to senescence and inflammation, including, but not limited to, IL-6, IL-8, MIP-1, IL-1b, IL-1a, eotaxin and IFNg. In some embodiments, such reduction can be a reduction of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an expected result of downregulating the expression and/or activity of USP16 in the cells of the disclosure is the associated increase in the expression of stem cell markers. Such stem cell markers include, but are not limited to, CD45RA, CD62, CCR7, TBX21, LEF1, TCF7, EOSOMES, IL7R, FOXPL ZEB2, BCL6, CD127, and CXCR3. In some embodiments, such increase can be an increase of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated reduction in sensitivity to senolytics. In some embodiments, such reduction can be a reduction of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated reduction in the observation of the senescence associated secretory phenotype (SASP). In some embodiments, such reduction can be a reduction of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated reduction of apoptosis. In some embodiments, such reduction can be a reduction of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated reduction of necrosis. In some embodiments, such reduction can be a reduction of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated increase in mitochondrial membrane potential. In some embodiments, such increase can be an increase of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

As is noted in the above section, an observed or expected result of downregulating the expression and/or activity of USP16 in the cells of the disclosure is the associated increase in cellular glutathione content. In some embodiments, such increase can be an increase of at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or even at least about 300%.

### Delivery Methods

The USP16 inhibitor may be delivered to a cell in numerous different ways. For example, the inhibitor (e.g., a small molecule) may be added directly to the media of the cell in culture. In some embodiments, the inhibitor may be delivered to the cell using a vector.

In some embodiments, the inhibitor may be delivered to the cell using a non-viral vector. Exemplary non-viral vectors include, but are not limited to, nanoparticles (e.g., polymeric nanoparticles), liposomes (e.g., cationic liposomes), cationic lipid-DNA complexes, lipid emulsions, calcium phosphate, polymer complexes, or combinations thereof. The non-viral vector may be used to package a double stranded DNA (dsDNA) (e.g., a plasmid), or a single stranded DNA (ssDNA). In some embodiments, non-viral vector may comprise a plasmid comprising a sequence encoding an inhibitor of USP16.

In some embodiments, the inhibitor may be delivered to the cell using a viral vector. For example, a nucleic acid sequence encoding the inhibitor may be packaged into a viral vector, and the viral vector may be subsequently used to transduce the cell. In some embodiments, the viral vectors of the instant disclosure are replication defective, or at least conditionally replication defective. Suitable viral vectors for use in the compositions and methods of the disclosure include, but are not limited to, retroviral vectors (e.g., lentiviral vectors), adenoviral vectors, and adeno-associated viral vectors (AAVs). In some embodiments, the viral vector is an AAV vector having a serotype selected from AAV1, AAV2, AAV3, AAV4, AAV5, AAV6, AAV7, AAV8, AAV9, AAV10, AAV11, AAV12, AAVrh.8, AAVrh.10, AAVrh32.33, AAVrh74, bovine AAV, and avian AAV. In some embodiments, the AAV vector is selected from any of the AAV vectors disclosed in Table 1 of WO 2019/028306, which is incorporated by reference herein in its entirety.

In some embodiments, the inhibitor may be delivered using a transposon system. Transposon systems have the capacity of stable genomic integration and long-lasting expression of transgene constructs in cells, including human cells. In some embodiments, the transposon system is the Sleeping Beauty system. The Sleeping Beauty system is composed of a Sleeping Beauty (SB) transposase and a transposon designed to insert specific sequences of DNA into genomes of vertebrate animals. As do other Tc1/mariner-type transposases, SB transposase inserts a transposon into a TA dinucleotide base pair in a recipient DNA sequence.

The inhibitor may also be delivered to the cell using electroporation. Electroporation briefly opens pores in the cell membrane, allowing passage of, for example, a DNA vector into the cell.

In some embodiments, the inhibitor is delivered alone, or in combination with one or more additional agents. In some embodiments, the one or more additional agents comprise a second inhibitor of USP16. In some embodiments, the one or more additional agents comprise a Wnt agonist. In some embodiments, the one or more additional agents comprise a R-spondin (Rspo) agonist. In some embodiments, an inhibitor of USP16 and a second agent are delivered simultaneously. In some embodiments, an inhibitor of USP16 and a second agent are delivered sequentially.

### Pharmaceutical Compositions

Also provided are pharmaceutical compositions comprising an inhibitor of USP16. In some embodiments, a pharmaceutically acceptable composition comprises an inhibitor of USP16 and one or more of a pharmaceutically acceptable carrier or excipient.

As used herein, “pharmaceutically acceptable carrier” or “pharmaceutical acceptable excipient” includes any material which, when combined with an inhibitor of USP16, allows the inhibitor of USP16 to retain biological activity. An excipient can give form or consistency, or act as a diluent. Suitable excipients include but are not limited to stabilizing agents, wetting and emulsifying agents, salts for varying osmolarity, encapsulating agents, buffers, and skin penetration enhancers. Examples include, but are not limited to, any of the standard pharmaceutical carriers/excipients such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 21st Ed. Mack Publishing, 2005).

### Methods of Treating

In some embodiments, a method of treating a subject in need thereof may comprise administering to the subject a therapeutically effective amount of an inhibitor of USP16. As used herein, the term “therapeutically effective amount” means the amount of an inhibitor that is sufficient to reduce the expression and/or activity of USP16 in a subject or in a cell.

In some embodiments, a method of treating a subject in need thereof may comprise administering to the subject a therapeutically effective number of cells that have been previously contacted with an inhibitor of USP16. In some embodiments, a method of treating a subject in need thereof may comprise administering to the subject a therapeutically effective number of cells that have been modified to downregulate USP16. As used herein, the term “therapeutically effective number” means the number of cells required to ameliorate or eliminate the symptoms of a disease or disorder in the subject. The therapeutically effective number of cells may be about 10², about 10³, about 10⁴, about 10⁵, about 10⁶, about 10⁷, about 10⁸, about 10⁹, about 10¹⁰, about 10¹¹, or about 10¹² cells, or more.

In some embodiments, the treatment is allogeneic. In other embodiments, the treatment is autologous. It is noted that additional genetic modifications may be introduced in order to prepare the cells for treatment, to minimize chances of rejection and the like.

In some embodiments, a method of treating a subject in need thereof may comprise administering to the subject a vector (e.g., a viral vector) comprising a sequence encoding an inhibitor of USP16. The viral vector may inhibit USP16 in one or more cell types in the subject in vivo.

A USP16 inhibitor or a cell modified to downregulate USP16 may be administered to the subject using various different administration routes, including oral, rectal, transmucosal, topical, transdermal, inhalation, intravenous, subcutaneous, intradermal, intramuscular, intra-articular, intrathecal, intraventricular, intravenous, intraperitoneal, intranasal, or intraocular routes of administration.

The inhibitor or the cell may be administered once, two times, three times, four times, five times, six times, seven times, eight times, nine times, ten times, or more to the subject. In some embodiments, the inhibitor or the cell may be administered to the subject at therapeutically effective intervals, e.g., once per day, once per week, once per month, once every 3 months, once every 6 months, once every year, etc.

The subject may be, for example, a rat, a dog, a mouse, a horse, a cat, a chicken, a non-human primate, or a human. In some embodiments, the subject is a human. The human may be, for example, less than about 5 years old, less than about 10 years old, less than about 20 years old, less than about 30 years old, less than about 40 years old, less than about 50 years old, less than about 60 years old, or greater than 60 years old. The subject may be male, or the subject may be female.

The subject may have, or may be suspected of having, one or more diseases or disorders. In some embodiments, the subject has cancer. The cancer may be, for example, leukemia, lymphoma, melanoma, multiple myeloma, pancreatic cancer, breast cancer, colon cancer, lung cancer, colorectal cancer or brain cancer. In some embodiments, the cancer may be a solid tumor. In some embodiments, the cancer may be a hematological malignancy. In some embodiments, the cancer may be a metastatic cancer.

In some embodiments, the subject may have, or may be suspected of having a cancer selected from: acute myeloid leukemia, adrenocortical carcinoma, AIDS-related cancers, AIDS-related lymphoma, anal cancer, appendix cancer, astrocytoma, basal cell carcinoma, bile duct cancer, bladder cancer, bone cancer (e.g., osteosarcoma/malignant fibrous histiocytoma), brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas/carcinoids, Burkitt lymphoma, cutaneous T-cell lymphoma, Hodgkin's lymphoma, non-Hodgkin's lymphoma, primary central nervous system lymphoma, carcinoid tumor, carcinoid tumor, central nervous system lymphoma, cerebellar astrocytoma, cerebral astrocytoma/Malignant glioma, cervical cancer, acute lymphocytic leukemia (ALL), chronic lymphocytic leukemia (CLL), acute myelogenous leukemia (AML), chronic myelogenous leukemia (CML), hairy cell leukemia, chronic myeloproliferative disorders, colon cancer, cutaneous T-cell lymphoma, desmoplastic small round cell tumor, endometrial cancer, ependymoma, esophageal cancer, Ewing's sarcoma, extracranial germ cell tumor, extragonadal germ cell tumor, extrahepatic bile duct cancer, eye cancer (e.g., intraocular melanoma, retinoblastoma), gallbladder cancer, gastric (stomach) cancer, gastrointestinal carcinoid tumor, gastrointestinal stromal tumor (GIST), germ cell tumor (extracranial, extragonadal, or ovarian), gestational trophoblastic tumor, glioma of the brain stem, childhood cerebral astrocytoma, gastric carcinoid, hairy cell leukemia, head and neck cancer, heart cancer, hepatocellular (liver) cancer, hodgkin lymphoma, hypopharyngeal cancer, hypothalamic and visual pathway glioma, intraocular melanoma, islet cell carcinoma, Kaposi sarcoma, kidney cancer (renal cell cancer), laryngeal cancer, lip and oral cavity cancer, liposarcoma, primary liver cancer, non-small cell lung cancer, small cell lung cancer, Waldenstrom macroglobulinemia, Merkel cell carcinoma, mesothelioma, mouth cancer, multiple endocrine neoplasia syndrome, multiple myeloma/plasma cell neoplasm, mycosis fungoides, myelodysplastic syndromes, myelodysplastic/myeloproliferative diseases, chronic myeloproliferative disorders, nasal cavity and paranasal sinus cancer, nasopharyngeal carcinoma, neuroblastoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer (e.g., islet cell pancreatic cancer), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, pituitary adenoma, plasma cell neoplasia, pleuropulmonary blastoma, primary central nervous system lymphoma, prostate cancer, rectal cancer, renal pelvis and ureter cancer, transitional cell cancer, retinoblastoma, rhabdomyosarcoma, salivary gland cancer, soft tissue sarcoma, uterine sarcoma, Sézary syndrome, non-melanoma skin cancer, melanoma, small cell lung cancer, small intestine cancer, soft tissue sarcoma, squamous cell carcinoma, squamous neck cancer with occult primary, stomach cancer, supratentorial primitive neuroectodermal tumor, T-cell lymphoma (cutaneous), testicular cancer, throat cancer, thymoma or thymic carcinoma, thyroid cancer, transitional cell cancer of the renal pelvis and ureter, trophoblastic tumor, urethral cancer, uterine cancer, uterine sarcoma, vaginal cancer, visual pathway and hypothalamic glioma, childhood, vulvar cancer, Waldenstrom macroglobulinemia, and Wilms tumor.

In some embodiments, the subject has a blood disorder, such as hemophilia A, hemophilia B, thalassemia, or anemia, etc.

Also provided are pharmaceutical kits for treating a subject suffering from a disease or disorder, the kit comprising cells modified to downregulate or knockout expression of USP16. In some embodiments, the cells are immune cells such as T-cells. In some embodiments, the cells are genetically modified cells. In some embodiments, the kit further comprises written instructions for administering the cells to the subject.

Also provided are kits for manufacturing a cell to be used for a cell-based therapy, the kit comprising an inhibitor of USP16. In some embodiments, the inhibitor of USP16 is a nucleic acid, a protein, a small molecule, or a large molecule. In some embodiments, the inhibitor of USP16 is a Cas nuclease, a TAL nuclease, a Zn finger nuclease, a RNAi molecule (e.g., small hairpin RNA (shRNA), a small interfering RNA (siRNA), a microRNA, an asymmetric interfering RNA), an antisense molecule, a phosphorothioate oligonucleotide, a DNA-RNA chimera, a morpholino oligo, a lhRNA, a miRNA embedded shRNA, a small internally segmented RNA, an antibody, or an exosome. In some embodiments, the kit further comprises written instructions for contacting the cells with the inhibitor of USP16.

Also provided are articles of manufacture for carrying out the methods. In some embodiments, the article of manufacture includes a plurality of containers, e.g., sealable containers, each individually comprising a unit dose of cells for administration to the subject, packaging material, and/or a label or package insert.

The foregoing is illustrative of the present invention, and is not to be construed as limiting thereof. The invention is defined by the following claims, with equivalents of the claims to be included therein.

### EXAMPLES

The following examples are included for illustrative purposes and are not intended to limit the scope of the invention.

### Example 1: CDKN2A Levels Increase During T Cell Differentiation

An in silico analyses was performed using previously-published microarray data (See Gattinoni L., et al, “A human memory T cell subset with stem cell-like properties,” Nature Medicine (2011); see also, “Expression data from human naïve (TN), stem cell memory (TSCM), central memory (TCM) and effector memory (TEM) CD8+ T cells,” Gene Expression Omnibus (GEO): GSE23321 (2011)). In this study, three healthy human blood donors provided lymphocyte-enriched apheresis blood after informed consent.

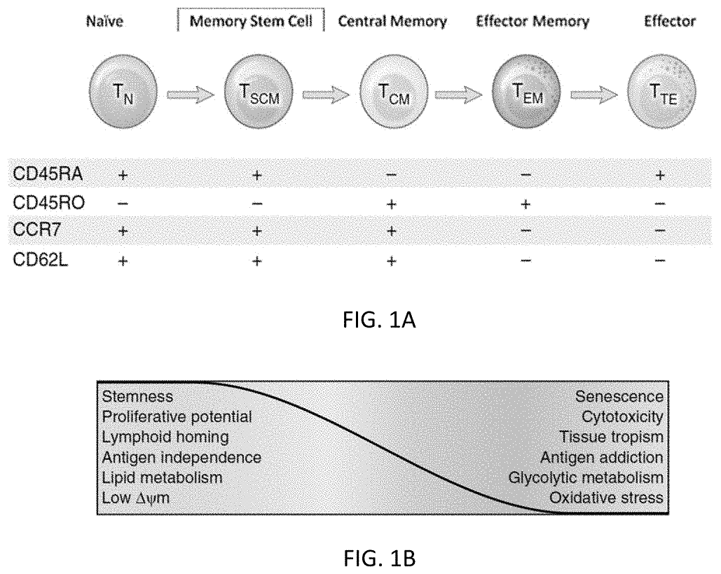

The expression levels of CDKN2A (probe: 8160441) in T cells at various stages of differentiation (FIG. 1) were analyzed using the GPL6244 platform. The analysis was performed using GEO2R.

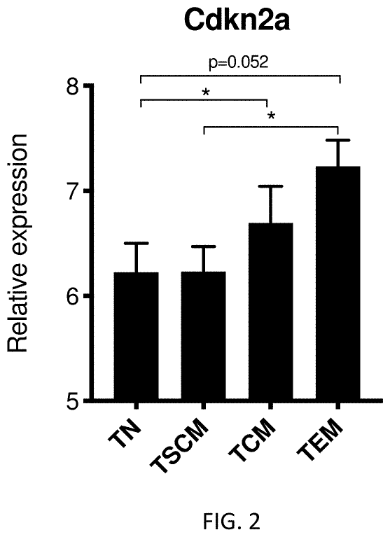

The results of the in silico analysis are provided in FIG. 2. Relative expression of CDKN2A was lower in naïve T cells (TN) and memory stem cells (TSCM), as compared to the central memory (TCM) and effector memory cells (TEM). This data demonstrates that CDKN2A levels increase during T cell differentiation.

### Example 2: In Vitro T Cell Activation Induces Cellular Aging and Reduces Stem Cell Markers

An in silico analyses was performed by using the online tool GEO2R (NCBI) to analyze previously-published, publicly available microarray data (See Cieri, et al, “IL-7 and IL-15 instruct the generation of human memory stem T cells from naive precursors,” Blood (2013); see also, Gene Expression Omnibus (GEO): GSE41909 (2012)). In this study, RNA was collected from unmanipulated purified T cells sorted for naive and central memory (CM) phenotype (pre-) or from T cell subsets that were in vitro activated and transduced (post-). In vitro generated subsets were kept in culture for 15 days in the presence of IL-7/IL-15 after activation.

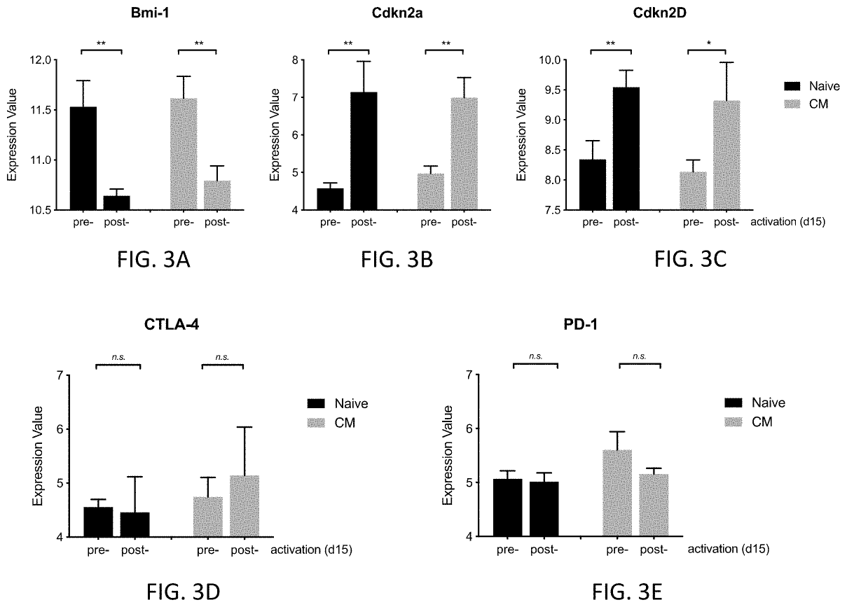

The results of the in silico analysis are provided in FIG. 3A-3E. In vitro activation of T cells induced the expression of the senescence markers CDKN2A (p16) (FIG. 3C) and CDKN2D (p19) (FIG. 3B) while suppressing the stem cell marker BMI1 (FIG. 3A). Interestingly, this event was not specific to a particular T cell subtype, and it was maintained in naïve and central memory T cells. Notably, expression of these senescence markers was observed well before exhaustion markers CTLA-4 (FIG. 3D) and PD-1 (FIG. 3E) start to be expressed.

Thus, these data show that reduction of stemness and induction of senescence is a very early event in the processes involved in the manufacturing of genetically modified T cells.

### Example 3: In Vitro T Cell Culture Conditions

Provided below are the conditions used to culture and activate T cells in vitro. T cells prepared according to this method were used in various Examples below.

On Day 0, buffycoats were obtained by a clinical facility, and PBMCs were isolated by density centrifugation using Ficoll™. CD3 enrichment was performed using magnetic beads, PanT cell isolation from Myltenyi Biotec. The T cells were activated with aCD³/aCD28 Dynabeads (3:1 ratio).

On Day 2, a first round of transduction was performed with lentivirus. On Day 3, a second round of transduction was performed with lentivirus.

On Day 6, the beads were removed. On Day 6-8, Flow cytometry sorting was performed with a FACSAria II (BD).

The cells were cultured in RPMI, 10% FBS, penicillin-streptomycin, and 100IU of IL-2. Cells were plated at a density of 0.3×10⁶ cells/ml until day 15, then 0.5×10⁶ cells/ml and after day 25 at 1×10⁶ cells/ml.

### Example 4: Downregulation of USP16 Expression in Primary T Cells Increases Proliferation and Reduces Cell Expiration

A short hairpin RNA (shRNA) directed against USP16 (shUSP16) was purchased from Dharmacon. Primary T cells were activated as described in Example 3 and transduced with a lentivirus encoding either the shUSP16 or a non-targeting shRNA (Ctrl) and sorted for GFP on day 8. The same number of cells were sorted in both groups. Between 0.15 and 0.5×10⁶ cells were plated post-sorting with a purity higher than 90% and counted every 2-4 days by Trypan Blue exclusion using Vi-CELL XR cell counter (Beckman Coulter). Proliferation was measured as expansion ratio compared to the initial cell seeding. Cells were considered expired when total T cell count was below 50,000 cells.

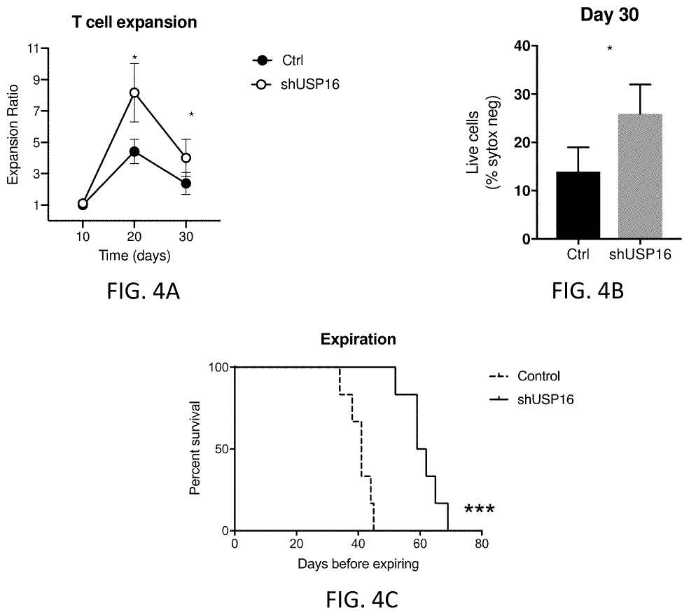

The experiment was repeated using primary T cells obtained from buffy coats of 8 different healthy donors. The graphs shown in FIG. 4A-4C show mean and SEM values, and the t-test was used to compare the two groups.

Downregulation of USP16 in T cells resulted in increased expansion upon T cell activation at day 20 (4.42 vs 8.17, p=0.0286, n=8) and day 30 (2.385 vs 4.014, p=0.037, n=8) (FIG. 4A). Flow cytometry analyses using Sytox staining also showed that at day 30 T cells downregulating USP16 were not only higher in number, but also more viable (13.96 vs 25.93, p=0.020, n=7) (FIG. 4B).

A T cell expiration assay (FIG. 4C) showed the strong effect of downregulation of USP16 expression in increasing T cell-persistence in vitro (median survival 41 vs 60.5 days, p=0.0005, n=6).

### Example 5: Downregulation of USP16 Expression in Primary T Cells does not Impair the CD4/CD8 Ratio

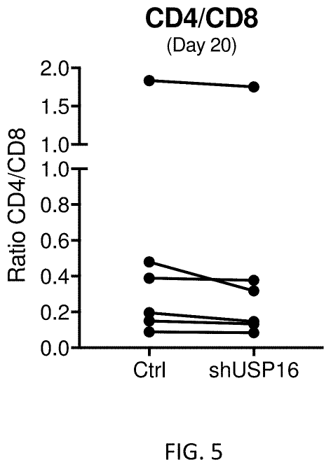

Flow cytometry analyses were performed at day 20 and the ratio of CD4 and CD8 was calculated. As shown in FIG. 5, the specific downregulation of USP16 expression had no effect on CD4/CD8 ratio (n=6; CD4 and CD8 percentages were calculated on Sytox-negative GFP⁺ T cells).

### Example 6: Navitoclax-Induced Cell Death Increases in T Cells During In Vitro Culture

Activated primary T cells were treated with the indicated amount of Navitoclax. Navitoclax is a senolytic drug selectively targeting senescent cells (Zhu et al., Aging Cell, 2016). After 72 hours, Sytox-negative T cells were counted by flow cytometry using MACSQuant (Myltenyi Biotec).

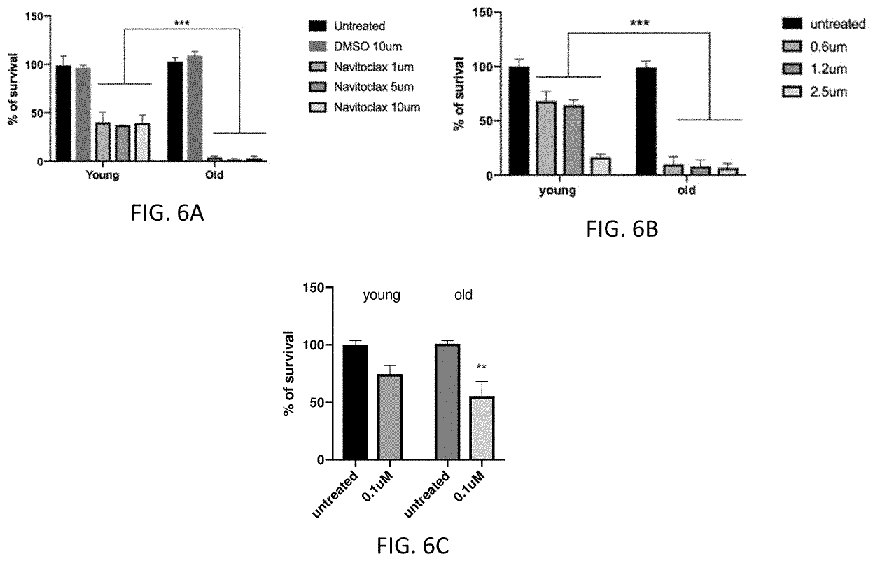

“Younger” T cells (day 12) are less sensitive to Navitoclax-induced cell death than “Older” T Cells (day 24). In each experiment two different primary T cell cultures were tested for each condition (four replicas for each condition). Three experiments testing different Navitoclax concentrations are shown in FIG. 6A-6C. DMSO is a negative control. Navitoclax (cat #HY-10087) was purchased from MCE.

### Example 7: T Cells Down-Regulating USP16 are Less Susceptible to Navitoclax Treatment

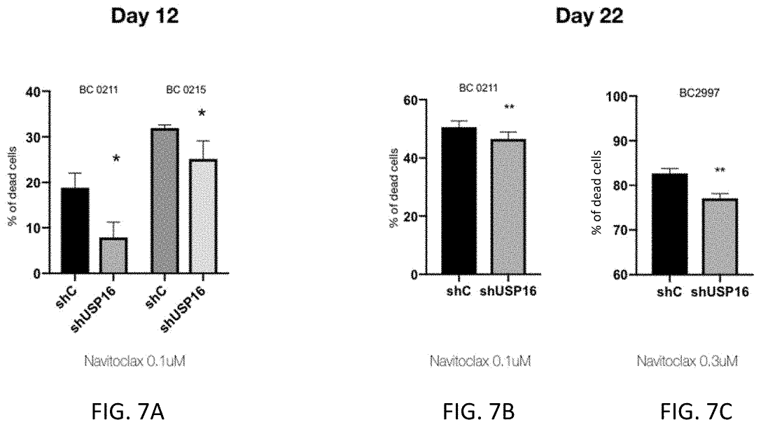

Primary T cells were activated as described (Example 3) and transduced with a lentivirus encoding either a shUSP16 or a non-targeting shRNA (shC). T cells were then treated with the indicated amount of Navitoclax. After 72 hours, Sytox-negative GFP+ cells were counted, and the numbers of dead cells were calculated as 100%−(#treated/#untreated) cells. Two different time points were tested (day 12, day 22) with two different primary T cell cultures (four replicas per each condition). As shown in FIG. 7A-7C, shUSP16 T cells are less sensitive to Navitoclax-induced cell death than their matching controls.

### Example 8: T Cell Self-Renewal is Reduced During In Vitro Culturing

An in vitro limiting dilution assay was used to determine the frequency of stem cells in a specific population. A range of cell concentration from 1000 to 4 cells per well (1:4 dilution) was determined to be sufficient to detect difference in T cell self-renewal capability. Here, it is demonstrated that in vitro T cell aging causes reduced self-renewal.

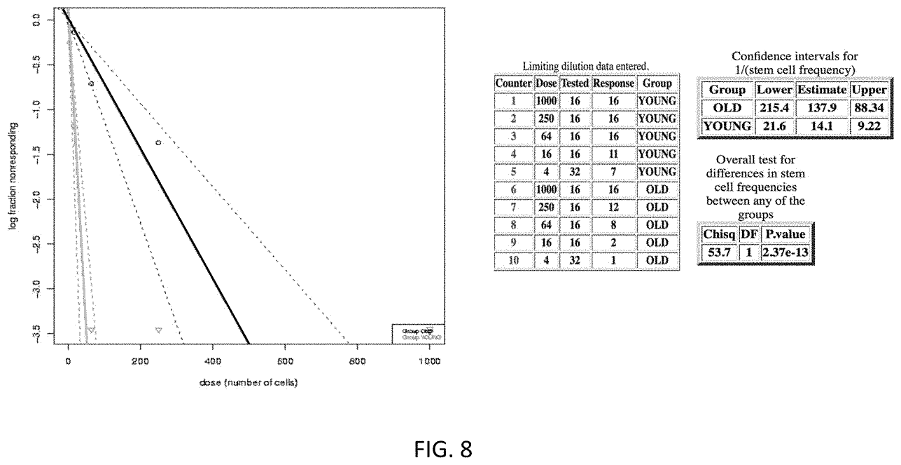

Younger T cells (day 8) were compared to older T cells (day 35). Cells were seeded at the specified concentration, and colony formation was assessed 2 weeks later. Fresh IL-2 was added every 3-4 days. Two primary T cells obtained from heathy donors were tested per every time point (eight replicates per cell concentration). The data were analyzed using the extreme limiting dilution analysis (ELDA) software (bioinf.wehi.edu.au/software/elda/). As shown in FIG. 8, older T cells have a reduced stem cell frequency (1/137.9) compared to younger cells (1/14.1, p=2.37e-13).

### Example 9: Downregulation of USP16 Expression Enhances In Vitro T Cell Self-Renewal

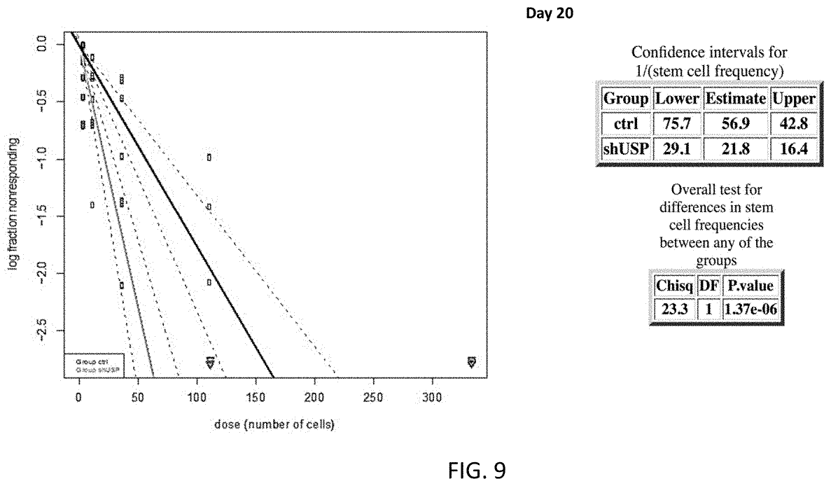

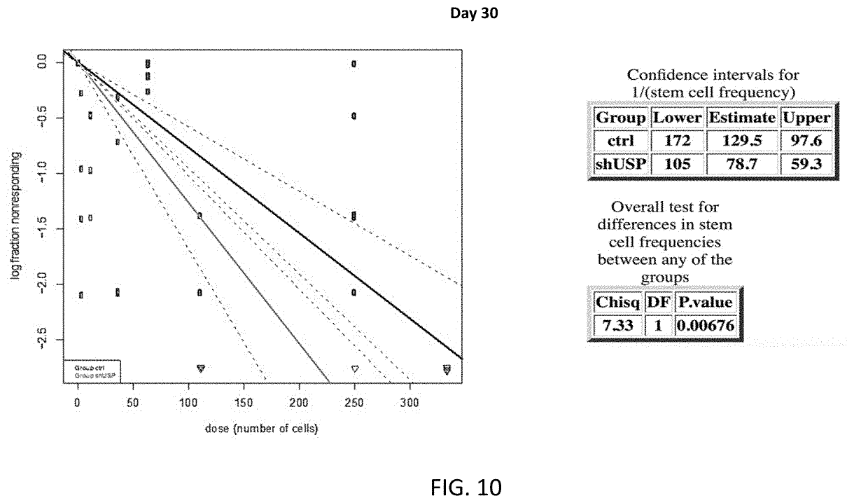

Primary T cells were activated as described (Example 3), transduced with a lentivirus encoding either a shUSP16 or a non-targeting shRNA (Ctrl) and sorted for GFP on day 8. Limiting dilution assays with a range of cell concentration from 1000 to 4 cells per well (1:3 dilution) were performed at day 20 (FIG. 9) and day 30 (FIG. 10) and colonies were counted two weeks after plating. Respectively, four and five primary T cell cultures were tested (eight replicates per cell concentration). Fresh IL-2 was added every 3-4 days. The data were analyzed using ELDA software.

Downregulation of USP16 expression significantly increases stem cell frequency (shCtrl vs shUSP Day 20: 1/56.9 vs 1/21.8, p=1.37e-6 and Day 30: 1/129.5 vs 78.8, p=0.00676).

### Example 10: Downregulation of USP16 Expression Increases Stem Cell Memory T Cells

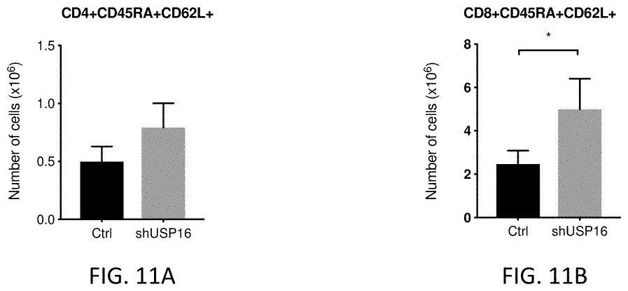

Primary T cells were activated as described (Example 3), transduced with a lentivirus encoding for either a shUSP16 or a non-targeting shRNA (Ctrl) and sorted for GFP on day 8. Cells were cultured in the presence of IL-2 and their phenotype was analyzed at day 20. Cells were stained for CD4-PerCP-5.5 or CD8-BV510, CD62L-Allophycocyanin, CD45RA-BV510 or PE-Cy7 (Biolegend). Naïve or stem cell memory T cells (T_{Na/SCM}) were identified as Sytox⁻ GFP⁺CD45RA⁺CD62L⁺. Number of T_{Na/SCM} was calculated as (#CD4 or CD8 T cells*T_{Na/SCM}percentage)/100. Flow cytometry was performed on the MACQuant (Myltenyi Biotec) and the data analyzed with FlowJo (shCtrl vs shUSP CD4: 0.4978 vs 0.7915, p=0.0673 and CD8: 2.469 vs 4.984, p=0.00392, n=7). Downregulation of USP16 expression increases the number of CD4+CD45RA+CD62L+(FIG. 11A) and CD8+CD45RA+CD62L+(FIG. 11B) cells in culture by 1.7 (p=0.0205) and 1.9 (p=0.0097) times, respectively.

### Example 11: T Cells with Downregulated USP16 Expression Maintain the Ability to Kill Tumor Cells In Vitro

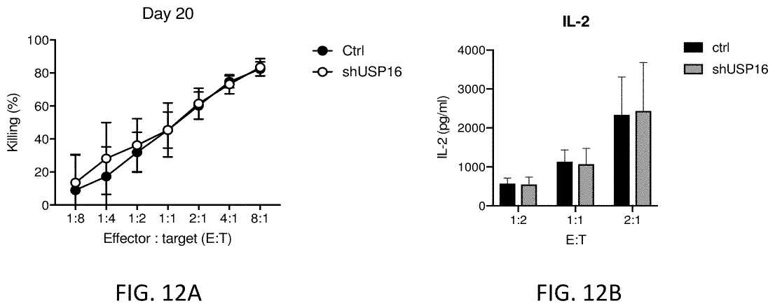

Cytotoxicity assays were performed. Briefly, 20 days old T cells were plated together with tumor cells at different Effector:Target (E:T) ratios and killing efficiency was measured after 72 hours by cell count using flow cytometry (MACSQuant, Myltenyi Biotec). Percentage of killing was measured as (#target-#residual target)*100/#target. NALM cells were used as target cells and infected with mRuby for identification by flow cytometry. Five different primary T cell cultures were tested (FIG. 12A). Cytotoxicity was tested also at day 31 and no differences were observed (data not shown).

IL-2 was measured by ELISA (Biolegend) 24 hours after cell seeding. As shown in FIG. 12B, downregulation of USP16 expression had no effect of IL-2 production upon antigen exposure.

### Example 12: The BMI-1/USP16/CDKN2A Pathways are Involved in CAR-T Cell Persistence in Humans

An in silico analyses was performed using previously-published microarray data (See Long, A. H., et al, “4-1BB costimulation ameliorates T cell exhaustion induced by tonic signaling of chimeric antigen receptors,” Nature Medicine (2015); see also, “Expression data from Chimeric Antigen Receptor (CAR) Expressing T cells,” Gene Expression Omnibus (GEO): GSE65856 (2015)). Human T cells isolated from healthy donors were transduced with non-tonically signaling CARs (CD19) or tonically signaling CARs, each with CD28z or 4-1BB costimulatory domains. The expression levels of CDKN2A, and BMI-1 were analyzed using the GPL570 platform. For CDKN2A, two probes were analyzed and expression level normalized on CD28 co-stimulus. The analyses were performed using GEO2R.

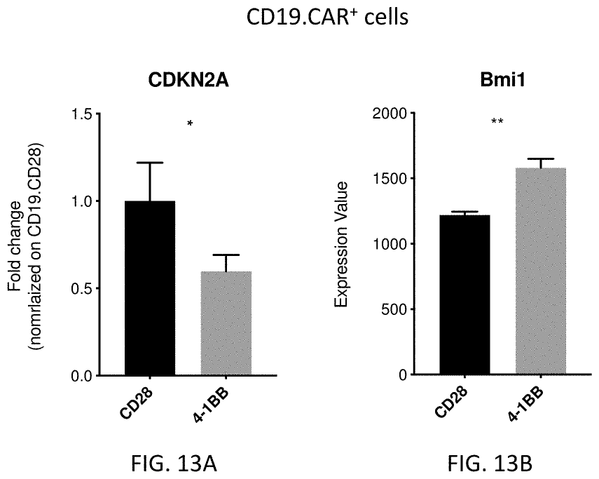

The 4-1BB stimulus has been shown to demonstrate higher persistence in CD19.CAR clinical trials comparing to CD28. In this analysis, more persistent cells expressed reduced levels of CDKN2A (FIG. 13A) and higher levels of BMI-1 (FIG. 13B), demonstrating the importance of this pathways in T cells also in the CAR-T setting.

### Example 13: An Alternative Set of shRNAs Targeting USP16 Effectively Downregulate USP16 and Inhibit Senescence Markers while Increasing Cell Proliferation

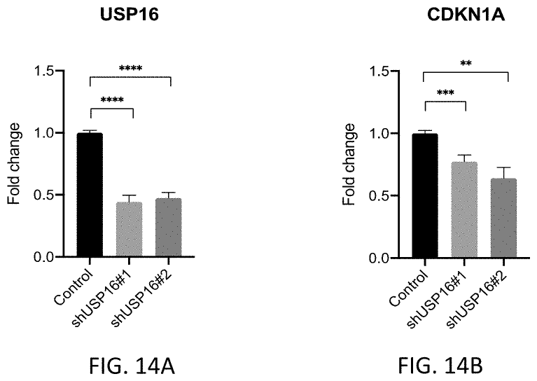

A new set of shRNAs were cloned into the pSIH backbone, where the shRNA expression is driven by the H1 promoter. The vector was also engineered to co-express GFP and Puromycin resistance through a T2A sequence. The new shRNA sequences were: shUSP16 #1 5′-GACTGTAAGACTGACAATAAA-3′ (SEQ ID NO: 8) and shUSP16 #2 5′-TATATCAGTTCACCCGTAAT-3′ (SEQ ID NO: 9). Primary T cells were activated as described (Example 3), transduced with a lentivirus encoding either a shUSP16 #1, shUSP16 #2 or a scrambled shRNA (Control) and cells were selected with puromycin at day 6. Between 1 and 1.25×10⁶ cells were plated post-puromycin selection with a purity higher than 90% and counted every 2-4 days by Trypan Blue exclusion using Vi-CELL XR cell counter (Beckman Coulter). qPCR was performed to verify downregulation of USP16 expression (FIG. 14A) and the expression of the senescent marker CDKN1A (FIG. 14B) at day 15. Proliferation was measured as fold increased compared to shControl at day 15 post-activation. The experiment was repeated using primary T cells obtained from buffy coats of 3 different healthy donors.

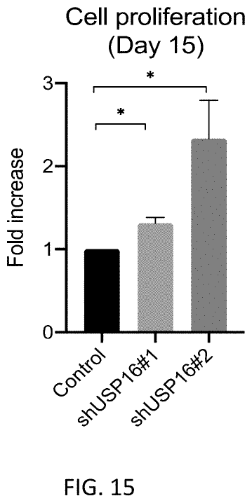

Downregulation of USP16 in T cells (Ctrl vs shUSP #1: 56%, p<0.0001 and Ctrl vs shUSP16 #2: 53%, p<0.0001) significantly decreases senescence marker CDKN1A (Ctrl vs shUSP #1: 23%, p=0.0009 and Ctrl vs shUSP16 #2: 37%, p=0.0014) and increases proliferation by about 1.31 and 2.33 folds (shUSP #1 and shUSP #2, respectively). FIG. 15 shows that down regulation of USP16 expression results in increased cell proliferation.

### Example 14: Downregulation of USP16 Expression Increases Signaling Through the WNT Pathway and Increases Stem Cell Memory T Cells Number and In Vitro Activity

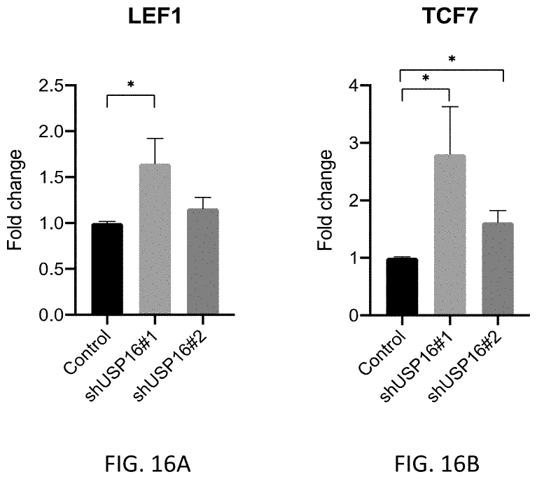

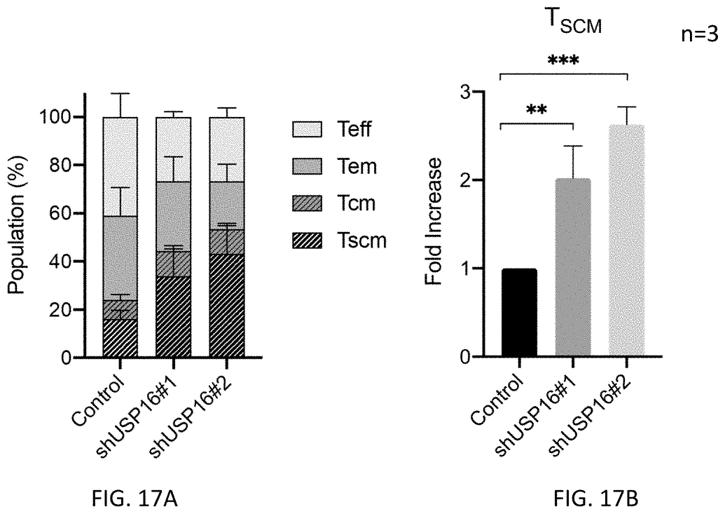

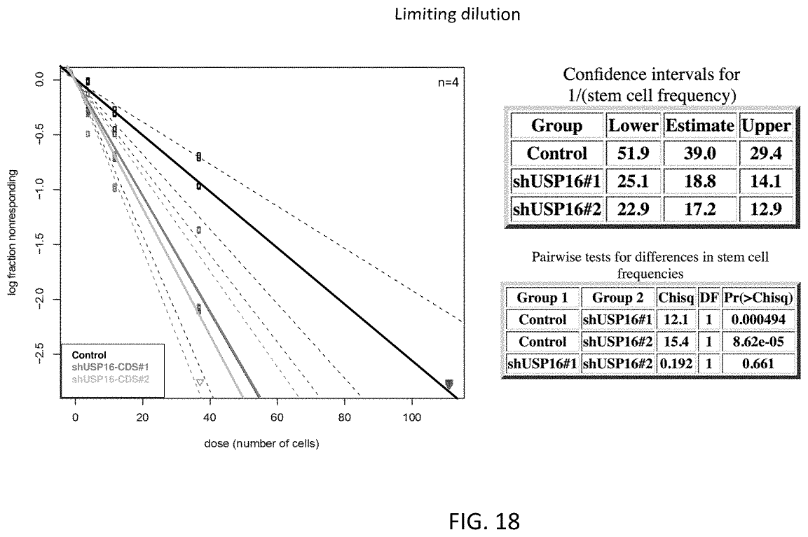

Primary T cells were activated as described (Example 3), transduced with a lentivirus encoding either a shUSP16 #1, shUSP16 #2 or a non-targeting shRNA (shCtrl) and cells were selected with puromycin at day 6. At day 15 cell were collected and analyzed by qPCR for the expression of LEF1 (FIG. 16A) and TCF7 (FIG. 16B), downstream regulators of the WNT pathway. At day 20, cells were stained for CD4-PE, CD8-BV510, CD62L-Allophycocyanin and CD45RA-APC-Cy7 (Biolegend). Stem cell memory T cells (T_{scm}) were identified as Sytox⁻ GFP⁺CD45RA⁺CD62L⁺, central memory T cells (T_{cm}) as Sytox⁻GFP⁺CD45RA⁻CD62L⁺, effector memory T cells (Tₑₘ) as Sytox⁻GFP⁺CD45RA⁻CD62L⁻, and terminal effector T cell (T_{eff}) as Sytox⁻GFP⁺CD45RA⁺CD62L⁻ (FIG. 17A). Cells were gated on the CD8 population. The specific increase of Tscm was shown as phenotype ratio (FIG. 17B).

Limiting dilution assays with a range of cell concentration from 1000 to 4 cells per well (1:3 dilution) were performed at day 20 (FIG. 18) and colonies were counted two weeks after plating. Four primary T cell cultures were tested (eight replicates per cell concentration). Fresh IL-2 was added every 3-4 days. The data were analyzed using ELDA software.

Downregulation of USP16 expression significantly increases the expression of LEF1 (fold increase over Control, shUSP16 #1: 1.650, p=0.0056; shUSP16 #2 displays a similar trend) and TCF7 (fold increase over Control, shUSP16 #1: 2.804, p=0.086; shUSP16 #2: 1.428, p=0.0467), demonstrating a higher activation of the WNT pathway. Downregulation of USP16 expression also significantly increases the stem cell frequency (fold increase over Control, shUSP16 #1: 2.022, p=0.0082; shUSP16 #2: 2.63, p=0.0001) and, most importantly, stem cell activity and self-renewal capacity (shCtrl vs shUSP #1 vs shUSP16 #2: 1/51.9 vs 1/25.1 vs 1/22.9, p=0.000494 and p=8.62e-05). Data are shown as mean+/−SEM.

### Example 15: Downregulation of USP16 Expression Decreases the Expression of the Exhaustion Marker CD69

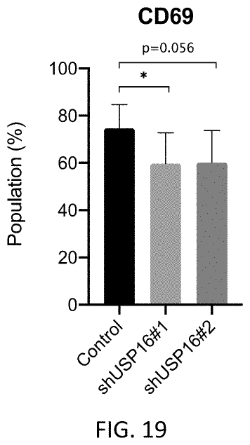

Primary T cells were activated as described (Example 3), transduced with a lentivirus encoding either a shUSP16 #1, shUSP16 #2 or a non-targeting shRNA (shCtrl) and cells were selected with puromycin at day 6. At day 20, T cells were stained for CD69-Allophycocyanin (Biolegend) and T cells analyzed by MACSQuant (Miltenyi Biotec) (FIG. 19). Three primary T cell cultures were analyzed.

USP16 Downregulation reduced the expression of the exhaustion marker CD69 (shCtrl vs shUSP #1: 41%, p=0.0443 and shCtrl vs shUSP16 #2: 40%, p=0.0569).

### Example 16: T Cells Downregulating USP16 Expression Maintain the Ability to Kill Tumor Cells In Vitro

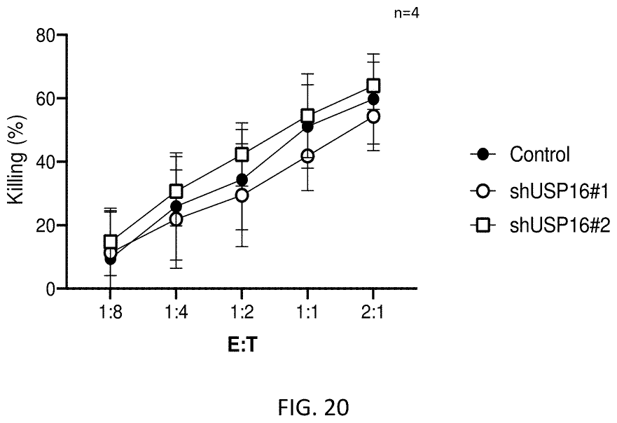

Cytotoxicity assays were performed. Briefly, 20 day old T cells were plated together with tumor cells at different Effector:Target (E:T) ratios and killing efficiency was measured after 72 hours by cell count using flow cytometry (MACSQuant, Miltenyi Biotec). Percentage of killing was measured as (#target−#residual target)*100/#target. NALM cells were used as target cells and infected with mRuby for identification by flow cytometry. Four different primary T cell co-cultures were tested (FIG. 20). downregulation of USP16 expression does not reduce the ability of T cells to kill target cells.

### Example 17: CRISPR-Mediated Knockout of USP16 Expression Increases Stem Cell Memory T Cells

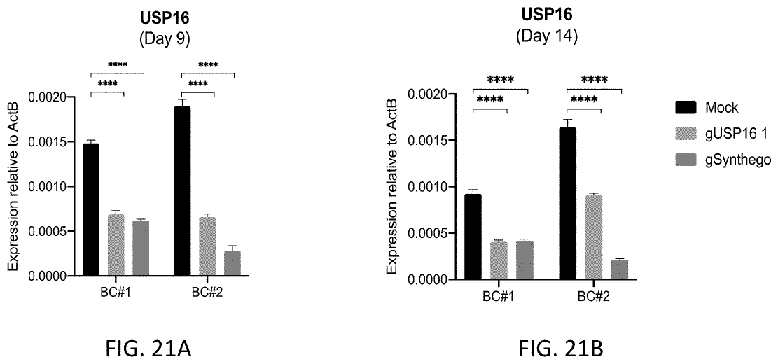

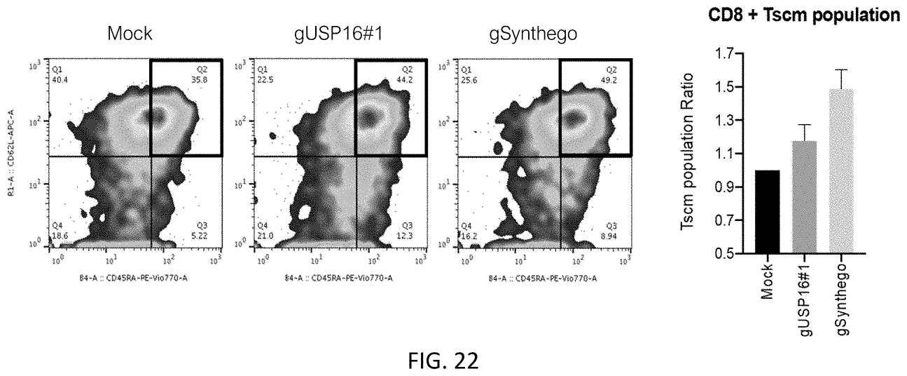

Primary T cells were activated as described (Example 3). At Day 6 post-activation, dynabeads were removed and T cells electroporated using the Neon Transfection System (ThermoFisher) with a 2.5:1 ratio sgRNA:Cas9. The experiment was performed following the protocol optimized for primary T cell by Synthego (CRISPR Editing Human Primary CD4+ T cells with RNPs using Neon Electroporation Protocol, 2018 version). The sgRNA used are the following: gUSP16 #1 5′-UGGCGUCAGAUAGUGCUUCA-3′ (SEQ ID NO: 10) and gSynthego that comprises 3 different sgRNAs (gRNA-A: 5′-GUGUGCAGACACAUUAGAAA 3′ (SEQ ID NO: 11); gRNA-B: 5′-UAUUGUCAGUCUUACAGUCU-3′ (SEQ ID NO: 12); gRNA-C: 5′-GUUUGGCUGUGUCUUAAAUG-3′ (SEQ ID NO: 13)). Control T cells, identified as Mock, were electroporated but no Cas9:sgRNA mixture was added. Cell were then assessed for USP16 expression by qPCR at day 9 (FIG. 21A) and day 14 (FIG. 21B) and an efficient knockout was observed in a portion of the population. At day 15 post-activation, cells were stained for CD4-PE, CD8-BV510, CD62L-Allophycocyanin and CD45RA-APC-Cy7 (Biolegend). Stem cell memory T cells (T_{scm}) were identified as Sytox⁻GFP⁺CD45RA⁺CD62L⁺ and graphed as ratio compared to mock cells (FIG. 22). The experiment was repeated using primary T cells obtained from buffy coats of 2 different healthy donors.

Downregulation of USP16 expression using CRISPR technology increases stem cell memory T cells by 1.178 and 1.49 (gUSP16# and gSynthego, respectively).

### Example 18: CRISPR-Mediated Downregulation of USP16 Expression does not Impair T Cell-Mediated Killing

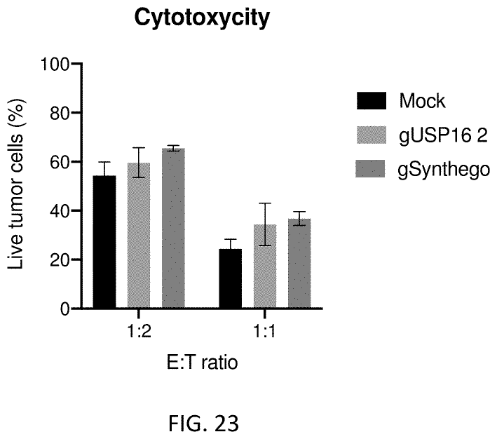

Cytotoxicity assays were performed. Briefly, 20 days old T cells were plated together with tumor cells at 1:1 and 1:2 Effector:Target (E:T) ratios and killing efficiency was measured after 72 hours by cell count using flow cytometry (MACSQuant, Myltenyi Biotec). Percentage of residual live cells was measured as (#residual target)*100/#target. NALM cells were used as target cells and infected with mRuby for identification by flow cytometry. Two different primary T cell cultures were tested (FIG. 23). CRISPR-mediated downregulation of USP16 expression does not alter T cell ability to kill tumor cells in vitro.

### Example 19: A Novel CAR Construct Engineered to Modulate USP16

Plasmids encoding for 2^{nd} generation CAR specific for CD19 (Lenti-EF1a-CD19(FMC63)-2nd-CAR(CD28)-EGFRt) or GD2 (Lenti-EF1a-GD2(14G2a)-2nd-CAR(CD28)-EGFRt) were purchased from Creative Biolabs. The CAR containing plasmids were engineered to co-express a shRNA targeting USP16. The shRNA sequences are: shUSP16 #1 5′-GACTGTAAGACTGACAATAAA-3′ (SEQ ID NO: 8) and shUSP16 #2 5′-TATATCAGTTCACCCGTAAT-3′ (SEQ ID NO: 9). Primary T cells were activated as described (Example 3), transduced with a lentivirus encoding for either a CD19.CAR or a GD2.CAR co-expressing shUSP16 #1, shUSP16 #2 or a scrambled shRNA (Control). Cells were cultured in IL-2 and sorted between days 6-13 for EGFRt. Briefly, cells were stained with biotin EGFR antibody (R&D) and magnetically sorted with anti-biotin or streptavidin conjugated beads (Miltenyi Biotec) using the LS columns (Miltenyi Biotec). The positive population was isolated and expanded in regular TC-treated plates or in G-Rex 6 well culture plate (Wilson Wolf). The G-Rex platform was used to achieve a rapid expansion of T cells for in vitro and in vivo applications.

### Example 20: CAR-T Cells Co-Expressing shRNA Targeting USP16 have a Proliferation Advantage

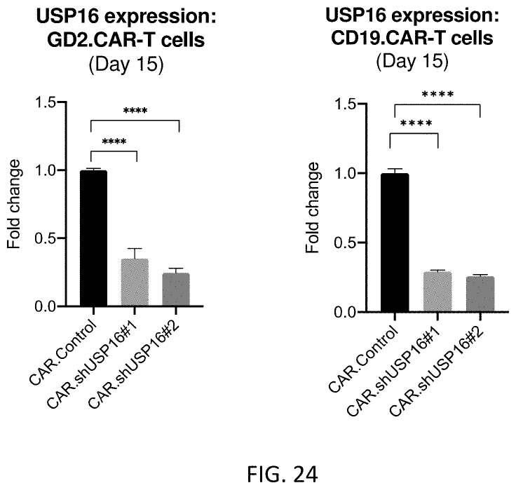

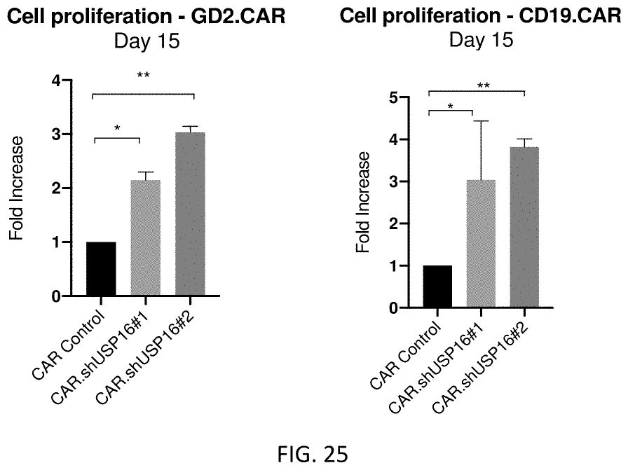

Primary T cells were activated as described (Example 3), transduced with a lentivirus encoding for either a CD19.CAR or a GD2.CAR co-expressing shUSP16 #1, shUSP16 #2 or a scrambled shRNA (Control). Cells were cultured in IL-2 and sorted between days 6-13 for EGFRt. Cells were plated post-sorting with a purity higher than 85% and counted every 2-4 days by Trypan Blue exclusion using Vi-CELL XR cell counter. Fifteen days post-activation both CD19.CAR and GD2.CAR T cells were collected, and qPCR was performed to verify downregulation of USP16 expression (FIG. 24) (fold change GD2.CAR (n=3): Control vs shUSP #1: 0.35, p<0.0001, and Control vs shUSP16 #2: 0.2452, p<0.0001, fold change CD19.CAR (n=4): Control vs shUSP #1: 0.2891, p<0.0001, and Control vs shUSP16 #2: 0.2566, p<0.0001). Proliferation was measured as fold increase compared to control cells at day 15 (FIG. 25).

USP16 expression downregulation in CAR-T cells enhances T cell expansion (fold change GD2.CAR (n=3): Control vs shUSP #1: 2.147, p=0.0267, and Control vs shUSP16 #2: 3.036, p=0.004, fold change CD19.CAR (n=4): Control vs shUSP #1: 2.259, p=0.0267, and Control vs shUSP16 #2: 3.036, p=0.004).

### Example 21: CAR-T Cells with Downregulation of USP16 Expression Show a Delay in Onset of Senescence and an Increase in Signaling Through the WNT Pathway

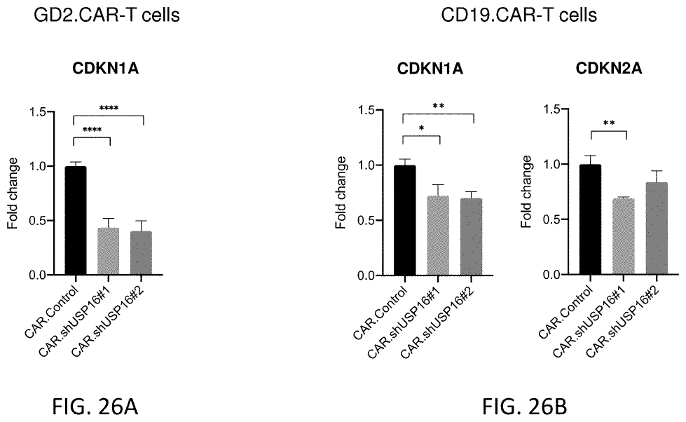

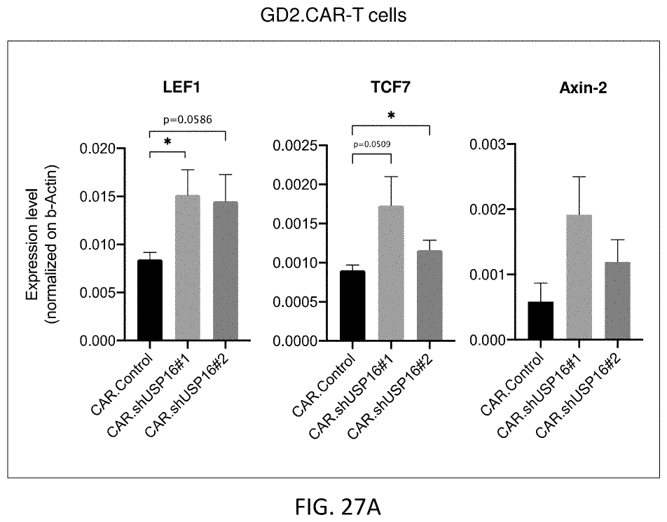

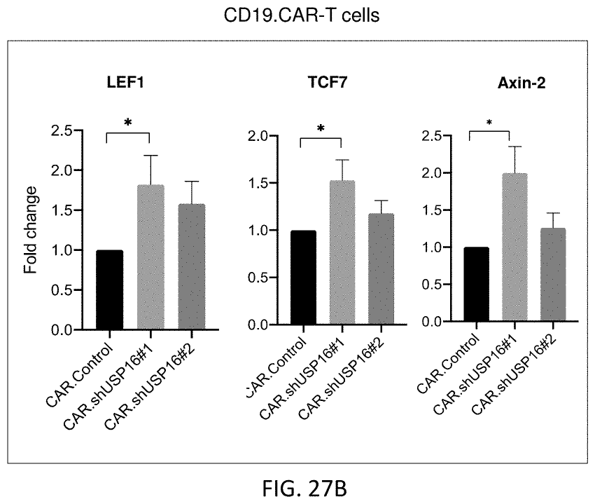

Primary T cells were activated as described (Example 3), transduced with a lentivirus encoding for either a CD19.CAR or a GD2.CAR co-expressing shUSP16 #1, shUSP16 #2 or a scrambled shRNA (Control). Fifteen days post-activation, T cells were collected and qPCR performed. TaqMan probes detecting CDKN1A, CDKN2A (FIGS. 26A and 26B) (fold change GD2.CAR (n=4): CDKN1A Control vs shUSP #1: 0.4360, p<0.0001, and Control vs shUSP16 #2: 3.036, p=0.004 respectively. Fold change CD19.CAR (n=4): CDKN2a, CDKN1A Control vs shUSP #1: 0.6906, p=0.0026; 0.7228, p=0.0297 and Control vs shUSP16 #2: 0.8385, p=0.2325; 0.7017, p=0.0021), TCF7, LEF1, and Axin-2 (FIGS. 27A and B) were analyzed in GD2.CAR and CD19.CAR-expressing cells, respectively. ACTB was used as internal control. (GD2.CAR (n=4) TCF7, LEF1 and Axin-2: Control vs shUSP #1: 1.914 p=0.0512; 1.795 p=0.0323; 3.269 p=0.15 and Control vs shUSP16 #2: 1.284 p=0.0494; 1.718 p=0.0586; 2.035p=0.235, respectively. Fold change CD19.CAR (n=4): TCF7, LEF1 and Axin-2: Control vs shUSP #1: 1.528, p=0.0277; 1.82, p=0.0449; 1.998, p=0.031 and Control vs shUSP16 #2: 1.18, p=0.2002; 1.579, p=0.0632; 1.261, p=0.2382). USP16 downregulation in CAR-T cells results in a decreased expression of senescence markers and an increase in the expression of genes associated with the activation/increased signaling of the WNT pathway, indicating its importance in delaying cellular senescence and increasing self-renewal.

### Example 22: Downregulation of USP16 Expression in CAR-T Cells Increases Stem Cell Memory T Cell Number and Functionality

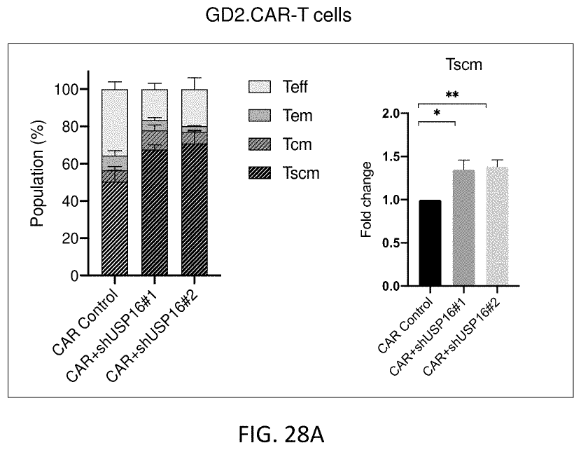

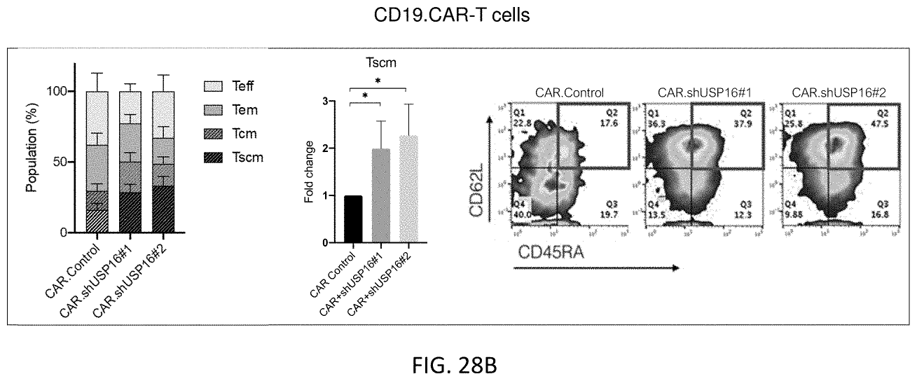

Primary T cells were activated as described (Example 3), transduced with a lentivirus encoding for either a CD19.CAR or a GD2.CAR co-expressing shUSP16 #1, shUSP16 #2 or a scrambled shRNA (Control). At day 15, GD2.CAR and CD19.CAR T cells were stained for EGFR-AF488 (R&D), CD3-PE, CD8-BV510, CD62L-Allophycocyanin and CD45RA-APC-Cy7 (Biolegend). Stem cell memory T cells (T_{scm}) were identified as FITC⁺CD45RA⁺CD62L⁺, central memory T cells (T_{cm}) as FITC⁺CD45RA⁻CD62L⁺, effector memory T cells (Tₑₘ) as FITC⁺CD45RA⁻CD62L⁻, and terminal effector T cell (T_{eff}) as FITC⁺CD45RA⁺CD62L⁻. Cells were gated on the CD8 population. The specific increase of Tscm was shown as phenotype ratio (FIGS. 28A and 28B).

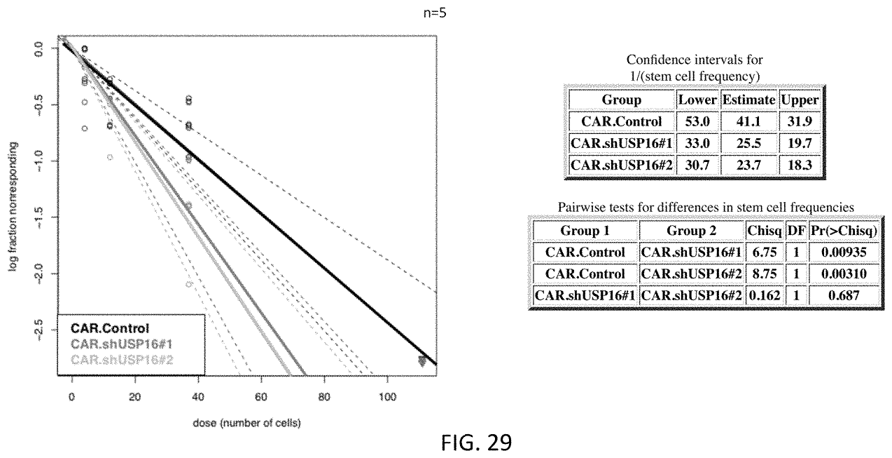

Limiting dilution assays with a range of cell concentration from 1000 to 4 cells per well (1:3 dilution) were performed at day 20 (FIG. 29) and colonies were counted two weeks after plating. Three CD19.CAR and two GD2.CAR primary T cell cultures were tested (eight replicates per cell concentration) and analyzed together. Fresh IL-2 was added every 3-4 days. The data were analyzed using ELDA software.

Downregulation of USP16 expression significantly increases the stem cell frequency (fold change CD19.CAR (n=5): Control vs shUSP #1: 1.994, p=0.032, and Control vs shUSP16 #2: 2.273, p=0.0214, fold change GD2.CAR (n=4): Control vs shUSP #1: 1.347, p=0.045, and Control vs shUSP16 #2: 1.383, p=0.0037) and, most importantly, stem cell activity and self-renewal capacity (CAR.Control vs CAR.shUSP #1 vs CAR.shUSP16 #2: 1/53 vs 1/33 vs 1/30.7, p=0.00935 and p=0.0031).

### Example 23: Downregulation of USP16 Expression in CAR-T Cells Increases Killing and T Cell Expansion in an In Vitro Cytotoxicity Assay

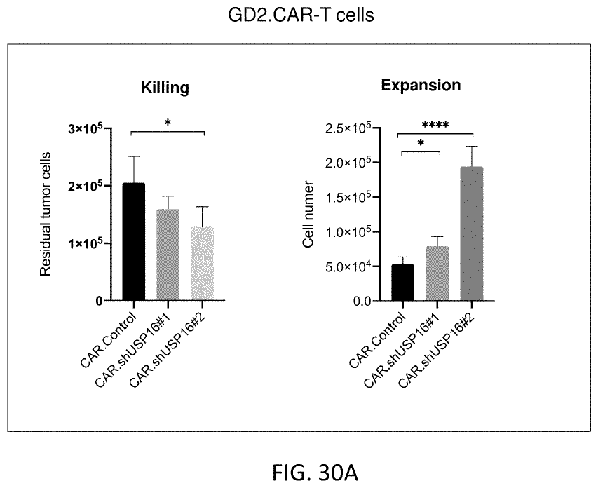

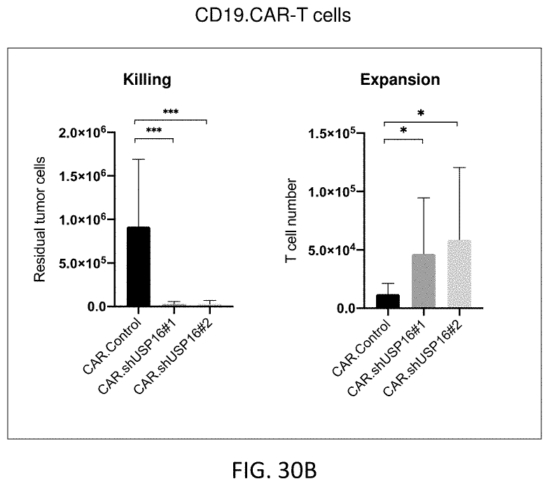

Primary T cells were activated as described (Example 3), transduced with a lentivirus encoding for either a CD19.CAR or a GD2.CAR co-expressing shUSP16 #1, shUSP16 #2 or a scrambled shRNA (Control). Cells were cultured in IL-2 and sorted between days 6-13 for EGFRt. At day 16 cytotoxicity assays were performed. Briefly, cells were counted and 100,000 T cells (adjusted for EGFR expression) were plated at 1:5 Effector:Target (E:T) ratio. At 72 hours, the mixture of tumor cells and T cells were collected, stained for CD3-PE, CD8-BV510, CD19- or GD2-APC, EGFR-AF488 and Sytox and counted by flow cytometry (MACSQuant, Miltenyi Biotec) (FIGS. 30A and 30B). Counting beads (CountBright Absolute Counting beads, Invitrogen) were added before acquisition and absolute number was calculated as following (number of cell events/number of bead events)×(assigned bead count of the lot (beads/50 ul)/volume of sample (ul))=concentration of sample as cells/ul). NALM cells were used as target cells for CD19.CAR T cells and CHLA-55 cells were used as target for GD2.CAR T cells. Three different primary T cell cultures were tested for CD19 and GD2 CAR.

Downregulation of USP16 expression in CAR-T cells improves their functionality upon antigen stimulation. CAR-T cells were able to kill a higher number of CHLA-55 and NALM-6 cells compared to the control. USP16 modulation also results in a significant increase in T cell expansion upon antigen stimulation (GD2.CAR (n=3) CHLA-55 residual cells and T cell number: Control vs shUSP #1 vs USP #2: 205317 vs 159531 vs 129186, p<0.0941; p=0.0265 and 53427 vs 79566 vs 194025, p=0.1462; p=0.0003. CD19.CAR (n=3): Control vs shUSP #1 vs USP #2: 918786 vs 29688 vs 28462, p<0.0001 and 12242 vs 46674 vs 58732, p=0.05; p=0.0404).

### Example 24: Downregulation of USP16 Expression in CAR-T Cells Increases Cellular Health and Reduces Cellular and Mitochondrial Stress

Primary T cells were activated as described (Example 3), and transduced with a lentivirus encoding for CD19.CAR co-expressing shUSP16 #1, shUSP16 #2 or a scrambled shRNA (Control). Cells were cultured in IL-2 and sorted between days 6-13 for EGFRt. At day 15 cells were stained and analyzed by MACSQuant for apoptosis, necrosis, mitochondrial membrane potential and glutathione content (GSH). The data generation was powered by Amberglass technology made available by Mojave Bio in the form of a dehydrated panel of reagents on a 96 well plate.

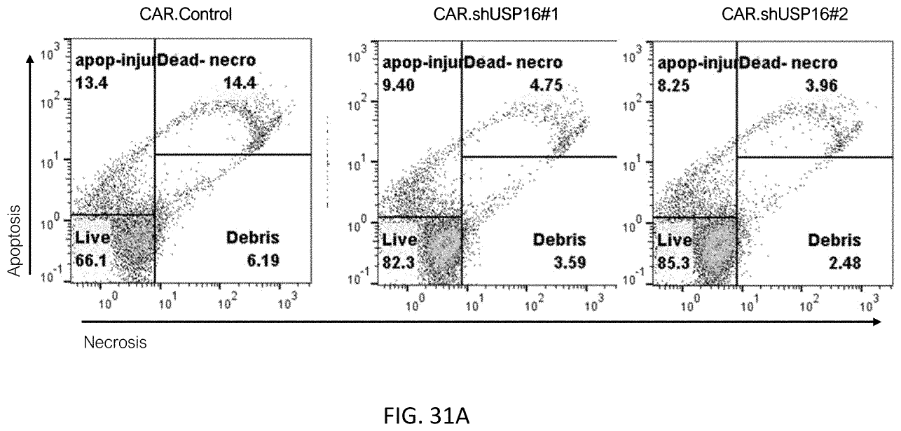

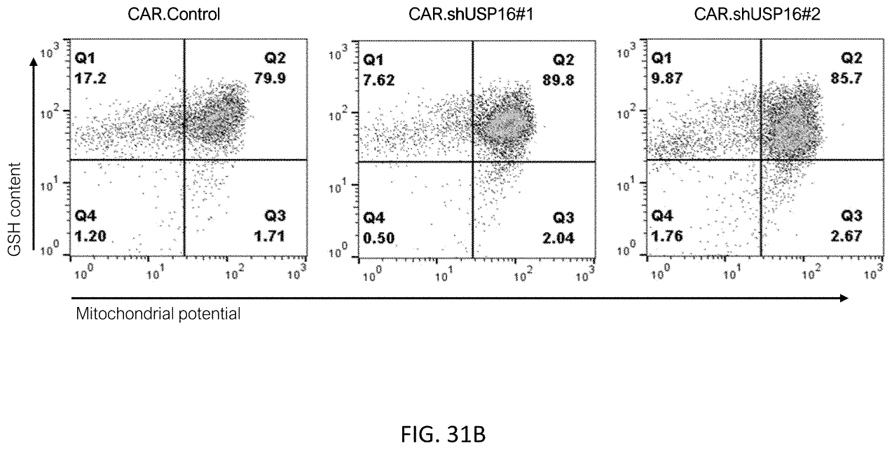

The plots in FIG. 31A show that downregulation of USP16 expression decreases apoptosis (Top left: Control vs shUSP16 #1 vs shUSP16 #2: 13.4 vs 9.40 vs 8.25) and necrosis (top right: Control vs shUSP16 #1 vs shUSP16 #2: 14.4 vs 4.75 vs 3.96).

The plots in FIG. 31B show the impact of downregulation of USP16 expression on cellular reactive oxygen species and stress. The figure displays (a) increased cellular health (high mitochondrial membrane potential and high GSH content, Q2: Control vs shUSP16 #1 vs shUSP16 #2: 79.9 vs 89.9 vs 85.7); and (b) reduced percentage of cells with low mitochondrial membrane potential (Q1: Control vs shUSP16 #1 vs shUSP16 #2: 17.2 vs 7.62 vs 9.87).

### Example 25: Downregulation of USP16 Expression in CAR-T Cells Reduces Exhaustion and Increases T Cell Killing Upon Multiple Challenges

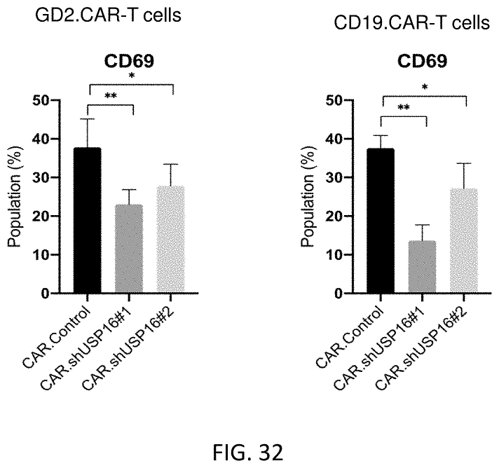

Primary T cells were activated as described (Example 3), transduced with a lentivirus encoding for either a CD19.CAR or a GD2.CAR co-expressing shUSP16 #1, shUSP16 #2 or a scrambled shRNA (Control). Cells were cultured in IL-2 and sorted between days 6-13 for EGFRt. At day 16, T cells were stained for exhaustion markers, including CD69. CD69 showed a reduced expression on CAR.T cell expressing the shRNA for USP16 (FIG. 32), further supporting that UPS16 modulation increases functionality and partially reduces T cell exhaustion (GD2.CAR (n=3) CD69%: Control vs shUSP #1 vs USP #2: 37.8 vs 23.05 vs 27.8, p=0.0051; p=0.0216, CD19.CAR (n=3) CD69%: Control vs shUSP #1 vs USP #2: 37.60 vs 13.70 vs 27.17, p=0.0015; p=0.0465).

At day 16, regular cytotoxicity assays were also performed (Example 22). Three days post-antigen stimulation, CD19.CAR T cells were stained for EGFR-AF488 (R&D), CD3-PE, CD8-BV510, PD-1-Allophycocyanin and Lag-3 and CTLA4 (Biolegend) and acquired by MACSQuant (Miltenyi Biotec). The co-cultures we also re-challenged with the addition of fresh tumor cells. T cell killing and expansion upon double tumor-challenge was assessed after three days.

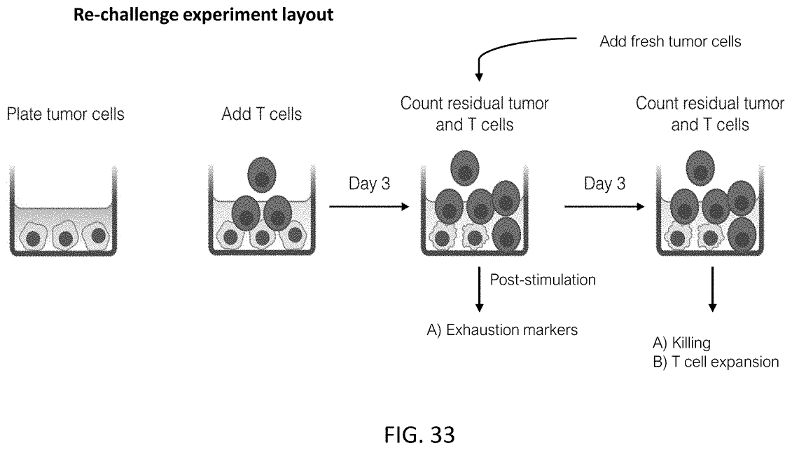

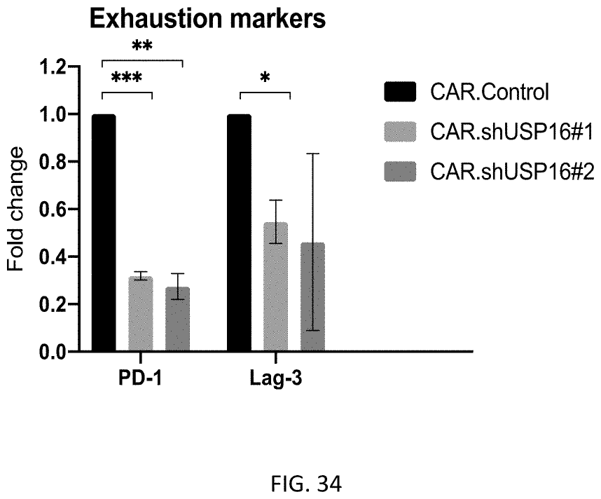

Sequential antigen stimulation drives exhaustion and results in reduced tumor killing and T cell expansion. Downregulation of USP16 expression in CAR.T cells is able to partially rescue exhaustion (FIG. 33). Data were analyzed by FlowJo. Three different primary T cell co-cultures expressing CAR.CD19 were analyzed, and the data are shown in the table below and in FIG. 34.

| | Fold change | P value |
|---|---|---|
| PD-1 | | |
| CAR.Control vs. CAR.shUSP16#1 | 0.3188 | <0.0001 |
| CAR.Control vs. CAR.shUSP16#2 | 0.2742 | 0.0029 |
| CAR.shUSP16#1 vs. CAR.shUSP16#2 | 0.2742 | 0.4729 |
| Lag-3 | | |
| CAR.Control vs. CAR.shUSP16#1 | 0.5467 | 0.0238 |
| CAR.Control vs. CAR.shUSP16#2 | 0.4613 | 0.2246 |
| CAR.shUSP16#1 vs. CAR.shUSP16#2 | 0.4613 | 0.9442 |

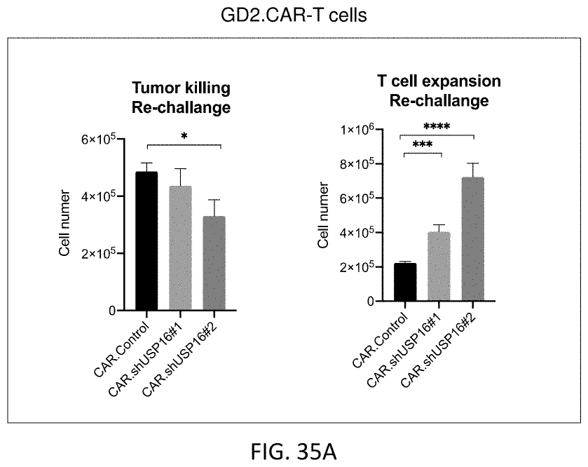

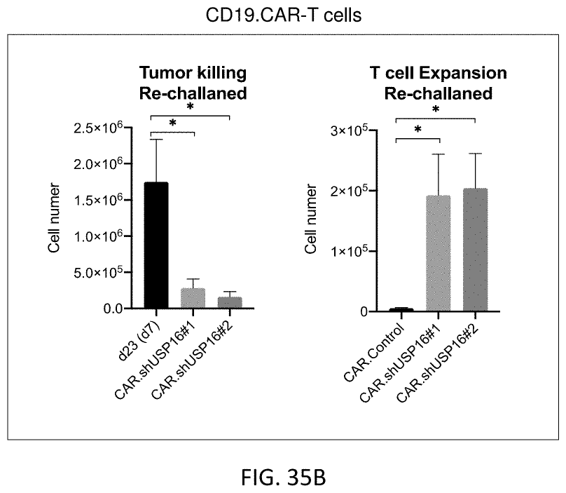

Downregulation of USP16 expression significantly increases T cell killing capability upon multiple tumor challenges (FIGS. 35A and 35B). On top of increasing killing function, downregulation of USP16 expression significantly augments T cell expansion after multiple tumor challenges, strongly demonstrating its important role in maintain young and fit T cells (GD2.CAR (n=3) CHLA-55 residual cells and T cell number: Control vs shUSP #1 vs USP #2: 486699 vs 436500 vs 330610, p<0.2839; p=0.0021 and 223281 vs 404709 vs 722634, p=0.0004; p<0.0001. CD19.CAR (n=3): Control vs shUSP #1 vs USP #2: 1749363 vs 282761 vs 160874, p=0.0242 and 5526 vs 192643 vs 204291, p=0.0385; p=0.171).

### Example 26: Co-Expression of shRNA Targeting USP16 Enhances GD2.CAR-T Anti-Tumor Activity In Vivo

Primary T cells were activated as described (Example 3), transduced with a lentivirus encoding for either a CD19.CAR or a GD2.CAR co-expressing shUSP16 #1, shUSP16 #2, or a scrambled shRNA (Control). Cells were cultured in IL-2 and sorted between days 6-13 for EGFRt. At day 15 cells were frozen down (to mimic the clinic setting) and thawed d−1 before injection in immunocompromised mice. Immunocompromised mice (NCG (NOD CRISPR Prkdc II2r Gamma) were provided by Charles River and NSG (NOD.Cg-Prkdc<scid>IL2rg<tm1Wj1>/SzJ) were purchased from Jackson Laboratory). CHLA-55 neuroblastoma cell lines were transduced with Gaussia Luciferase and one million cells were injected via tail vein at d−7 (day minus 7).

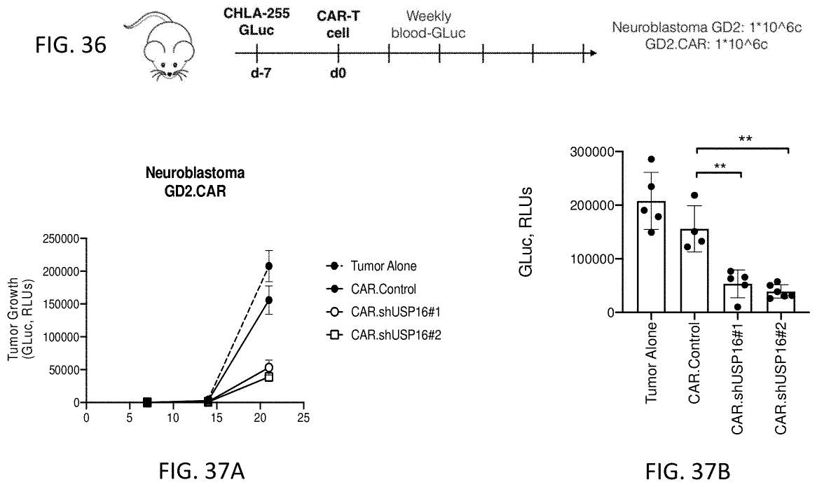

One million of GD2.CAR T cells were infused via tail vain at day 0 and mice were weighted, and blood was collected every week (FIG. 36). Tumor growth was monitor by weekly assessment of circulating luciferase. Briefly, blood was collected and subsequently analyzed for luciferase expression using Nanolight Technologies Luciferase detection Kit. Luciferase expression (calculated as GLuc, RLU (Relative Luminescence Unit)) in the blood correlates with tumor engraftment. CHLA-55 tumor growth over time is shown in FIG. 37A and CHLA-55 engraftment at the time of sacrifice is shown in FIG. 37B.

Downregulation of USP16 expression significantly increased CAR T cell killing function in vivo. The experiment was repeated with another T cell co-culture with similar results (n=4-6, Control vs shUSP #1 vs USP #2: 156009 vs 53279 vs 38968, p=0.0015; p=0.0003).

### Example 27: Co-Expression of shRNA Targeting USP16 Enhances CD19.CAR-T Anti-Tumor Activity In Vivo

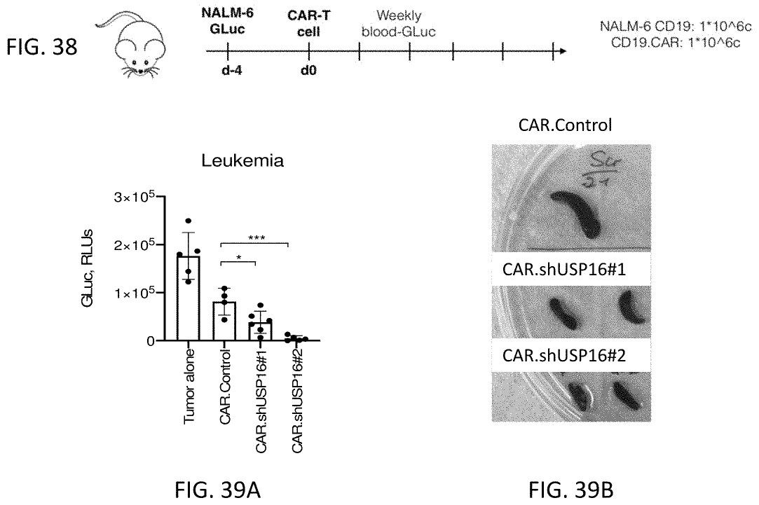

Primary T cells were activated and prepared, as in Example 26. NALM leukemia cell line was transduced with Gaussia Luciferase and one million cells were injected via tail vein at d−4. One million of CD19.CAR T cells were then infused via tail vein at d=0 and mice were weighted, and blood was collected every week (FIG. 38). Tumor growth was monitor by weekly assessment of circulating luciferase. Briefly, blood was collected and subsequently analyzed for luciferase expression using Nanolight Technologies Luciferase detection Kit. Luciferase expression (calculated as GLuc, RLU (Relative Luminescence Unit)) in the blood correlates with tumor engraftment. NALM engraftment is shown in FIG. 39A. As NALM cells engraft primarily in the spleen, at the time of sacrifice, the spleens were collected and photographed (FIG. 39B), the size of the spleen directly correlates to the amount of tumor infiltrated in the organ.

USP16 expression downregulation significantly increases CART cell killing function in vivo (n=4-6, Control vs shUSP #1 vs USP #2: 81507 vs 38603 vs 5169, p=0.0132; p=0.0003).

### Example 28: Transient Downregulation of USP16 Expression

Primary T cells are activated as described (Example 3). At Day 6 (+/−4 days) post-activation, dynabeads are removed and T cells electroporated using the Neon Transfection System (ThermoFisher). A working solution of 80 nM is used and the NEON settings are specified in Example 17. The electroporated cells are cultured in IL-2 and electroporated for a second time at day 10 (+/−4 days). At day 15 cells are collected, RNA extracted, and cells are analyzed for the expression of senescence genes, including CDKN1A, CDKN2a, CDKN2D and self-renewing genes. Cells are also stained for CD4-PerCP-5.5 or CD8-BV510, CD62L-Allophycocyanin, CD45RA-BV510 or PE-Cy7 (Biolegend). Stem cell memory T cells (Tscm) are identified as Sytox-CD45RA+CD62L+. Cells are also analyzed for exhaustion markers, including CD69 and tested for killing and expansion capability upon single and multiple stimulation. At day 20 limiting dilution assays are performed.

Expected results: T cells downregulating USP16 by means of a transient siRNA delivery is expected to result in a delayed onset of cellular senescence and a higher number and/or percentage of Tscm cells. It is further expected that these Tscm cells will perform better in a limiting dilution assay. T cells downregulating USP16 by means of a siRNA are also expected to have a higher cytotoxicity capability in vitro and in vivo (in immunocompromised mice, NCG or NSG injected with tumors) and a higher proliferation potential upon antigen triggering. Also, it is expected that T cell exhaustion might be delayed. Similar results are expected to be obtained in cells that have been edited or modified, e.g. CAR-T cells, TCR cells, and gene edited cells (e.g. with CRISPR).

### Example 29: Downregulation of USP16 Expression and its Effect on T Cell Resistance to Immunosuppressive Microenvironments

PMSCs isolated from buffycoats (Example 3) are used as source of CD8 T cells and monocytes. Briefly, CD8 T cells are magnetically isolated and activated for 6 days with a CD3/CD28, transduced with a lentivirus encoding for a scrambled (control) or a USP16 targeting shRNA and cultured in the presence of IL-2. Autologous MDCSs are differentiated starting form CD14⁺ cells (magnetically isolated from PBMCs) cultured in the presence of GM-CSF and IL-6 (long/ml) for 7 days. At this time, MDSCs are co-cultured with CF SE-labelled autologous CD8 T cells for 3 days. CSFE dilution, cytokines and phenotype are assessed at this time.

Expected results: While MDSCs strongly suppress T cell proliferation, it is expected that when USP16 expression is downregulated in T cells, the MDSC-mediated suppression will be less effective, that is T cells in which USP16 expression is downregulated will be less sensitive to an immunosuppressive environment.

Similar results are expected when T cell proliferation is suppressed by the co-culture with Tregs. To test this, briefly, autologous Treg, identified as CD4⁺CD25⁺CD127^{low}, are isolated from PBMCs via FACS-sorting or magnetic isolation and added to CD8 T cells (see above) at different ratios (1:1, 1:0.5, 1:0.25, Effector-to-TReg, etc.). After 6-day co-culture the percentage of divided precursors are assessed, e.g. by flow cytometry. Suppression is calculated by comparing the percentage of divided cells in the absence or presence of Treg cells or MDSCs (myeloid-derived suppressor cells). Similar results are expected to be obtained in cells that have been edited or modified, e.g. CAR-T cells, TCR cells, and gene edited cells (e.g. with CRISPR).

### Example 30: Downregulation of USP16 Expression and its Effect on the Susceptibility of T Cells to Tumor-Induced Senescence

Primary T cells are activated as described (Example 3), transduced with a lentivirus encoding for a shRNA targeting USP16 or with either a CD19.CAR or a GD2.CAR co-expressing shUSP16 #1, shUSP16 #2 or a scrambled shRNA (Control). Cells are sorted for EGFRt or selected by puromycin. To induce senescent T cells, tumor cell lines (e.g. 012SCC, CHLA-4, MG-63.3 MCF-7, HCT-116, and MEL-624) are cultured for 24 hours, then T lymphocytes (control or knocked down for USP16) are added at different tumor:T-cell ratios. Cells can be incubated from 6 h up to 5 days. T cells are then collected, washed, and directly analyzed or cultured for an additional 7 days in complete medium; no additional cytokines are added. Cells are then collected and analyzed for: exhaustion and memory markers (Flow cytometry), the differential expression of senescence and self-renewing genes or proteins (including CDKN2a, CDKN1A, CDKN2a, LEF-1, TCF7, Axin2, p27, p53), cell number and viability, and SA-β-Gal staining.

Expected results: it is expected that USP16 downregulation will make T cells less susceptible to tumor-induced senescence, since USP16 silenced T cells have higher viability, less senescence, increase self-renewal and they exhibit an enhanced ability to efficiently expand and kill tumor cells.

While the invention has been described in conjunction with the detailed description thereof, the foregoing description is intended to illustrate and not limit the scope of the invention, which is defined by the scope of the appended claims. Other aspects, advantages, and modifications are within the scope of the following.

1. A method of modulating cellular aging comprising contacting a cell with an inhibitor of USP16, wherein the cell is a blood cell.

2-137. (canceled)

138. A blood cell modified to downregulate expression of USP16.

139-161. (canceled)

162. A method of preparing an immune cell for adoptive cell therapy, the method comprising contacting the immune cell with an inhibitor of USP16.

163. The method of claim 162, wherein the immune cell is a T cell.

164. The method of claim 162, wherein the cell is a NK cell.

165. The method of claim 162, wherein the immune cell is not a stem cell.

166. The method of claim 162, wherein the immune cell is a genetically modified immune cell.

167. The method of claim 166, wherein the immune cell is genetically modified to express an exogenous protein.

168. The method of claim 167, wherein the immune cell is genetically modified to express a T cell receptor (TCR).

169. The method of claim 167, wherein the immune cell is genetically modified to express a chimeric antigen receptor (CAR).

170. The method of claim 169, wherein the CAR recognizes CD19, CD20, CD22, CD30, CD33, CD70, CD123, CD138, CD171, glypican-3, kappa immunoglobulin, ROR1, GD2, CD44v6, HER2, NY-ESO-1, BCMA, CD22, MSLN, CEA, EGFR, EGFRvIII, VEGFR2, IL-13, IL13Ra2, Lewis Y antigen, mesothelin, FAP, PSMA, or a combination thereof.

171. The method of claim 169, wherein the CAR is a dual-targeting CAR, an inhibitory CAR, an inducible CAR, a synNotch CAR, an iCAR, a drug-inducible CAR, or an adapter CAR.

172. The method of claim 169, wherein the CAR co-expresses a cytokine or a cytokine receptor, a suicide gene, an anti-exhaustion protein, a shRNA, a siRNA, or a gRNA.

173. The method of claim 162, wherein the inhibitor of USP16 is a nucleic acid.

174. The method of claim 162, wherein the inhibitor of USP16 is a protein.

175. The method of claim 162, wherein the inhibitor of USP16 is a small molecule.

176. The method of claim 162, wherein the inhibitor of USP16 is a large molecule.

177. The method of any claim 162, wherein the inhibitor is a RNAi molecule.

178. The method of claim 177, wherein the RNAi molecule is a shRNA, an siRNA, a microRNA, or an asymmetric interfering RNA.

179. The method of claim 162, wherein the inhibitor of USP16 is an antisense molecule, a phosphorothioate oligonucleotide, a DNA-RNA chimera, a morpholino oligo, a lhRNA, a miRNA embedded shRNA, a small internally segmented RNA, an antibody, an exosome, or a histone modifier.

180. The method of claim 162, wherein in the inhibitor of USP16 is delivered to the cell using a viral vector or a non-viral vector.

181. The method of claim 180, wherein the viral vector is a lentiviral vector or an adeno-associated viral vector.

182. The method of claim 180, wherein the non-viral vector is a liposome or a nanoparticle.

183. The method of claim 162, wherein the inhibitor of USP16 is delivered to the cell using a transposon system.

184. The method of claim 162, wherein the inhibitor of USP16 results in at least a 10% inhibition of expression and/or activity of USP16.

185. The method of claim 162, wherein the adoptive cell therapy is an immunotherapy.

186. The method of claim 185, wherein the immunotherapy is an autologous immunotherapy.

187. The method of claim 185, wherein the immunotherapy is an allogeneic immunotherapy.

188. The method of claim 162, wherein contacting the immune cell with an inhibitor of USP16 results in one or more of:
(a) increasing in vivo persistence;
(b) reducing cell exhaustion;
(c) increasing cellular proliferation;
(d) enhancing signaling through the WNT pathway;
(e) maintaining or increasing in vivo cell killing;
(f) increasing anti-tumor activity;
(g) maintaining or increasing Naïve or Central Memory phenotype;
(h) increasing the expression of stem cell markers;
(i) reducing production of reactive oxygen species (ROS);
(j) increasing in vivo engraftment; and
(k) preventing, delaying, or reversing the onset of senescence.

189. A method of treating a disease or disorder, the method comprising administering to a subject in need thereof a therapeutically effective amount of a cell produced using the method of claim 162.


SEQUENCE LISTING (13 sequences)

SEQ ID NO: 1 - protein, 823 aa, Homo sapiens
MGKKRTKGKTVPIDDSSETLEPVCRHIRKGLEQGNLKKALVNVEWNICQDCKTDNKVKDKAEEETEEKPSVWLCLKCGHQGCGRNSQEQHALKHYLTPRSEPHCLVLSLDNWSVWCYVCDNEVQYCSSNQLGQVVDYVRKQASITTPKPAEKDNGNIELENKKLEKESKNEQEREKKENMAKENPPMNSPCQITVKGLSNLGNTCFFNAVMQNLSQTPVLRELLKEVKMSGTIVKIEPPDLALTEPLEINLEPPGPLTLAMSQFLNEMQETKKGVVTPKELFSQVCKKAVRFKGYQQQDSQELLRYLLDGMRAEEHQRVSKGILKAFGNSTEKLDEELKNKVKDYEKKKSMPSFVDRIFGGELTSMIMCDQCRTVSLVHESFLDLSLPVLDDQSGKKSVNDKNLKKTVEDEDQDSEEEKDNDSYIKERSDIPSGTSKHLQKKAKKQAKKQAKNQRRQQKIQGKVLHLNDICTIDHPEDSEYEAEMSLQGEVNIKSNHISQEGVMHKEYCVNQKDLNGQAKMIESVTDNQKSTEEVDMKNINMDNDLEVLTSSPTRNLNGAYLTEGSNGEVDISNGFKNLNLNAALHPDEINIEILNDSHTPGTKVYEVVNEDPETAFCTLANREVFNTDECSIQHCLYQFTRNEKLRDANKLLCEVCTRRQCNGPKANIKGERKHVYTNAKKQMLISLAPPVLTLHLKRFQQAGFNLRKVNKHIKFPEILDLAPFCTLKCKNVAEENTRVLYSLYGVVEHSGTMRSGHYTAYAKARTANSHLSNLVLHGDIPQDFEMESKGQWFHISDTHVQAVPTTKVLNSQAYLLFYERIL

SEQ ID NO: 2 - DNA, 19 bp, Unknown
  USP16 shRNA target sequence
tccagaaggaatatcactt

SEQ ID NO: 3 - protein, 823 aa, Artificial Sequence; identical to SEQ ID NO: 1
  USP16 protein variant

SEQ ID NO: 4 - protein, 823 aa, Artificial Sequence; identical to SEQ ID NO: 1
  USP16 protein variant

SEQ ID NO: 5 - protein, 822 aa, Artificial Sequence
  USP16 protein variant
MGKKRTKGKTVPIDDSSETLEPVCRHIRKGLEQGNLKKALVNVEWNICQDCKTDNKVKDKAEEETEEKPSVWLCLKCGHQGCGRNSQEQHALKHYLTPRSEPHCLVLSLDNWSVWCYVCDNEVQYCSSNQLGQVVDYVRKQASITTPKPEKDNGNIELENKKLEKESKNEQEREKKENMAKENPPMNSPCQITVKGLSNLGNTCFFNAVMQNLSQTPVLRELLKEVKMSGTIVKIEPPDLALTEPLEINLEPPGPLTLAMSQFLNEMQETKKGVVTPKELFSQVCKKAVRFKGYQQQDSQELLRYLLDGMRAEEHQRVSKGILKAFGNSTEKLDEELKNKVKDYEKKKSMPSFVDRIFGGELTSMIMCDQCRTVSLVHESFLDLSLPVLDDQSGKKSVNDKNLKKTVEDEDQDSEEEKDNDSYIKERSDIPSGTSKHLQKKAKKQAKKQAKNQRRQQKIQGKVLHLNDICTIDHPEDSEYEAEMSLQGEVNIKSNHISQEGVMHKEYCVNQKDLNGQAKMIESVTDNQKSTEEVDMKNINMDNDLEVLTSSPTRNLNGAYLTEGSNGEVDISNGFKNLNLNAALHPDEINIEILNDSHTPGTKVYEVVNEDPETAFCTLANREVFNTDECSIQHCLYQFTRNEKLRDANKLLCEVCTRRQCNGPKANIKGERKHVYTNAKKQMLISLAPPVLTLHLKRFQQAGFNLRKVNKHIKFPEILDLAPFCTLKCKNVAEENTRVLYSLYGVVEHSGTMRSGHYTAYAKARTANSHLSNLVLHGDIPQDFEMESKGQWFHISDTHVQAVPTTKVLNSQAYLLFYERIL

SEQ ID NO: 6 - protein, 823 aa, Artificial Sequence; identical to SEQ ID NO: 1
  USP16 protein variant

SEQ ID NO: 7 - protein, 107 aa, Artificial Sequence
  USP16 protein variant
MGKKRTKGKTVPIDDSSETLEPVCRHIRKGLEQGNLKKALVNVEWNICQDCKTDNKVKDKAEEETEEKPSVWLCLKCGHQGCGRNSQEQHALKHYLTPRSEPHCLVL

SEQ ID NO: 8 - DNA, 21 bp, Unknown
  shRNA sequence shUSP16 1
gactgtaagactgacaataaa

SEQ ID NO: 9 - DNA, 20 bp, Unknown
  shRNA sequence shUSP16 2
tatatcagttcacccgtaat

SEQ ID NO: 10 - RNA, 20 nt, Unknown
  sgRNA gUSP16 1
uggcgucagauagugcuuca

SEQ ID NO: 11 - RNA, 20 nt, Unknown
  sgRNA gRNA-A
gugugcagacacauuagaaa

SEQ ID NO: 12 - RNA, 20 nt, Unknown
  sgRNA gRNA-B
uauugucagucuuacagucu

SEQ ID NO: 13 - RNA, 20 nt, Unknown
  sgRNA gRNA-C
guuuggcugugucuuaaaug